SEQUENCE LISTING (57 sequences)

SEQ ID NO: 1 - protein, 25 aa, synthetic construct
  VARIANT 2: note = X can be any amino acid
  VARIANT 3: note = X can be any amino acid
  VARIANT 5: note = X can be any amino acid
  VARIANT 6: note = X can be any amino acid
  VARIANT 7: note = X can be any amino acid
  SITE 9: note = This residue may be independently present or absent
  SITE 20..25: note = This region may be independently present or absent in its entirety
AXXIXXXAMGVADLIKKFESISKEE

SEQ ID NO: 2 - protein, 25 aa, synthetic construct
  VARIANT 2: note = X can be any amino acid
  VARIANT 3: note = X can be any amino acid
  VARIANT 5: note = X can be any amino acid
  VARIANT 6: note = X can be any amino acid
  VARIANT 7: note = X can be any amino acid
AXXIXXXAMGVADLIKKFESISKEE

SEQ ID NO: 3 - protein, 25 aa, synthetic construct
AEEIDEAAMGVADLIKKFESISKEE

SEQ ID NO: 4 - protein, 25 aa, synthetic construct
AEEIDEAAMEVADLIKKFESISKEE

SEQ ID NO: 5 - protein, 25 aa, synthetic construct
AEEIDEAAMGAADLIKKFESISKEE

SEQ ID NO: 6 - protein, 25 aa, synthetic construct
AEEIDEAAMAVADLIKKFESISKEE

SEQ ID NO: 7 - protein, 25 aa, synthetic construct
AENIDEAAMGVADLIKKFESISKEE

SEQ ID NO: 8 - protein, 25 aa, synthetic construct
AENIDEAAMEVADLIKKFESISKEE

SEQ ID NO: 9 - protein, 25 aa, synthetic construct
AENIDEAAMGAADLIKKFESISKEE

SEQ ID NO: 10 - protein, 25 aa, synthetic construct
AENIDEAAMAVADLIKKFESISKEE

SEQ ID NO: 11 - protein, 21 aa, synthetic construct
AEEIMGVADLIKKFESISKEE

SEQ ID NO: 12 - protein, 21 aa, synthetic construct
AENIMGVADLIKKFESISKEE

SEQ ID NO: 13 - protein, 135 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKT

SEQ ID NO: 14 - protein, 21 aa, synthetic construct
  VARIANT 2: note = X can be any amino acid
  VARIANT 3: note = X can be any amino acid
  SITE 5: note = This residue may be independently present or absent
  SITE 16..21: note = This region may be independently present or absent in its entirety
AXXIMGVADLIKKFESISKEE

SEQ ID NO: 15 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIDEAAMGVADLIKKFESISKEE

SEQ ID NO: 16 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIDEAAMGVADLIKKFESISKEE

SEQ ID NO: 17 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIDEAAMEVADLIKKFESISKEE

SEQ ID NO: 18 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIDEAAMEVADLIKKFESISKEE

SEQ ID NO: 19 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIDEAAMGAADLIKKFESISKEE

SEQ ID NO: 20 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIDEAAMGAADLIKKFESISKEE

SEQ ID NO: 21 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIDEAAMAVADLIKKFESISKEE

SEQ ID NO: 22 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIDEAAMAVADLIKKFESISKEE

SEQ ID NO: 23 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIDEAAMGVADLIKKFESISKEE

SEQ ID NO: 24 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAMGVADLIKKFESISKEE

SEQ ID NO: 25 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIDEAAMEVADLIKKFESISKEE

SEQ ID NO: 26 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAMEVADLIKKFESISKEE

SEQ ID NO: 27 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIDEAAMGAADLIKKFESISKEE

SEQ ID NO: 28 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAMGAADLIKKFESISKEE

SEQ ID NO: 29 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIDEAAMAVADLIKKFESISKEE

SEQ ID NO: 30 - protein, 160 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAMAVADLIKKFESISKEE

SEQ ID NO: 31 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIMGVADLIKKFESISKEE

SEQ ID NO: 32 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIMEVADLIKKFESISKEE

SEQ ID NO: 33 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIMGAADLIKKFESISKEE

SEQ ID NO: 34 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAEEIMAVADLIKKFESISKEE

SEQ ID NO: 35 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIMGVADLIKKFESISKEE

SEQ ID NO: 36 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIMEVADLIKKFESISKEE

SEQ ID NO: 37 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIMGAADLIKKFESISKEE

SEQ ID NO: 38 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLAKKTAENIMAVADLIKKFESISKEE

SEQ ID NO: 39 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIMGVADLIKKFESISKEE

SEQ ID NO: 40 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIMEVADLIKKFESISKEE

SEQ ID NO: 41 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIMGAADLIKKFESISKEE

SEQ ID NO: 42 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAEEIMAVADLIKKFESISKEE

SEQ ID NO: 43 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIMGVADLIKKFESISKEE

SEQ ID NO: 44 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIMEVADLIKKFESISKEE

SEQ ID NO: 45 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIMGAADLIKKFESISKEE

SEQ ID NO: 46 - protein, 156 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIMAVADLIKKFESISKEE

SEQ ID NO: 47 - protein, 17 aa, synthetic construct
  SITE 1: note = This residue may be independently present or absent
  SITE 12..17: note = This region may be independently present or absent in its entirety
MGVADLIKKFESISKEE

SEQ ID NO: 48 - protein, 17 aa, synthetic construct
MGVADLIKKFESISKEE

SEQ ID NO: 49 - protein, 17 aa, synthetic construct
MEVADLIKKFESISKEE

SEQ ID NO: 50 - protein, 17 aa, synthetic construct
MGAADLIKKFESISKEE

SEQ ID NO: 51 - protein, 17 aa, synthetic construct
MAVADLIKKFESISKEE

SEQ ID NO: 52 - protein, 6 aa, synthetic construct
SISKEE

SEQ ID NO: 53 - protein, 9 aa, synthetic construct
GGSGGSGGS

SEQ ID NO: 54 - protein, 11 aa, Avena sativa
AENIDEAAKEL

SEQ ID NO: 55 - protein, 11 aa, synthetic construct
AEEIDEAAKEL

SEQ ID NO: 56 - protein, 11 aa, synthetic construct
AXXIXXXAXXL

SEQ ID NO: 57 - protein, 6 aa, synthetic construct
HHHHHH

(19) United States
(12) Patent Application Publication
(10) Pub. No.: US 2024/0410901 A1
(43) Pub. Date: Dec. 12, 2024

(54) METHODS AND COMPOSITIONS COMPRISING ACTIN BINDING PROTEINS

(71) Applicant: THE UNIVERSITY OF CHICAGO, Chicago, IL (US)

(72) Inventors: Tobin SOSNICK, Chicago, IL (US); Kourtney KROLL, Chicago, IL (US); Ronald ROCK, Chicago, IL (US); Alexander FRENCH, Chicago, IL (US)

(73) Assignee: THE UNIVERSITY OF CHICAGO, Chicago, IL (US)

(21) Appl. No.: 18/698,606

(22) PCT Filed: Oct. 7, 2022
(86) PCT No.: PCT/US2022/077754

§ 371 Date: Apr. 4, 2024

Related U.S. Application Data
(60) Provisional application No. 63/253,420, filed on Oct. 7, 2021.

(51) Int. Cl. G01N 33/68; C07K 16/18; G01N 33/543

(52) U.S. Cl. CPC G01N 33/6887; C07K 16/18; G01N 33/54386; G01N 2333/4712

(57) ABSTRACT

The current disclosure describes light switchable actin binding protein (ABP) built from a photosensitive protein and an ABP. This is accomplished by creating an ABP that is caged and has a weak affinity to actin in the dark and uncaged with a strong affinity for actin in the light. These light-switchable polypeptides can recruit other actin binding proteins of interest to actin filaments spatiotemporally and reversibly. Aspects of the disclosure relate to polypeptides that are useful for labeling actin fibers. Accordingly, the disclosure relates to a polypeptide comprising the amino acid sequence: AXXIXXXA(M)nGVADLIKKFE(X′)n′ (SEQ ID NO:1) or an amino acid sequence with at least 80% sequence identity to SEQ ID NO:1, wherein X is any amino acid, n and n′ are each independently selected from 0 and 1, and X′ is equal to SISKEE.

### CROSS-REFERENCE TO RELATED APPLICATIONS

This application claims the benefit of priority of U.S. Provisional Patent Application No. 63/253,420 filed Oct. 7, 2021, which is hereby incorporated by reference in its entirety.

### GOVERNMENT SUPPORT CLAUSE

This invention was made with government support under grant numbers EB009412, GM055694 and GM124272 awarded by the National Institutes of Health. The government has certain rights in the invention.

### BACKGROUND OF THE INVENTION

### I. Field of the Invention

This invention relates to the field of molecular biology.

### II. Background

Cytoskeletal structure and dynamics are key for cells to function. Not only does the cytoskeleton give static support to the cell, its self-organization and restructuring are critical for cytokinesis, muscle movement, lamellipodia driven cell movement and more. Although both spatial and temporal organization are vital to how the actin network functions in cells, currently there is a general lack of tools that allows for the recapitulation of these natural events in vitro and in vivo. Therefore, there is a need in the art for additional tools to image actin networks.

### SUMMARY OF THE INVENTION

The current disclosure fulfills the need in the art by describing light switchable actin binding protein (ABP) built from a photosensitive protein and an ABP. This is accomplished by creating an ABP that is caged and has a weak affinity to actin in the dark and uncaged with a strong affinity for actin in the light. These light-switchable polypeptides can recruit other actin binding proteins of interest to actin filaments spatiotemporally and reversibly.

Aspects of the disclosure relate to polypeptides that are useful for labeling actin fibers. Accordingly, the disclosure relates to a polypeptide comprising the amino acid sequence: AXXIXXXA(M)nGVADLIKKFE(X′)n′ (SEQ ID NO:1) or an amino acid sequence with at least 80% sequence identity to SEQ ID NO:1, wherein X is any amino acid, n and n′ are each independently selected from 0 and 1, and X′ is equal to SISKEE. Aspects also relate to a polypeptide comprising the amino acid sequence: AXXI(M)nGVADLIKKFE(X′)n′ (SEQ ID NO: 14) or an amino acid sequence with at least 80% sequence identity to SEQ ID NO:14, wherein X is any amino acid, n and n′ are each independently selected from 0 and 1, and X′ is equal to SISKEE. In some aspects, the disclosure relates to a polypeptide comprising the amino acid sequence: AXXIXXXA(M)nGVADLIKKFE(X′)n′ (SEQ ID NO:1) or an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 1, wherein X is any amino acid, n and n′ are each independently selected from 0 and 1, and X′ is equal to SISKEE. Aspects also relate to a polypeptide comprising the amino acid sequence: AXXI(M)nGVADLIKKFE(X′)n′ (SEQ ID NO:14) or an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:14, wherein X is any amino acid, n and n′ are each independently selected from 0 and 1, and X′ is equal to SISKEE. Also disclosed are nucleic acid(s) encoding the polypeptides of the disclosure, expression vectors comprising nucleic acids of the disclosure, and host cells comprising polypeptides, nucleic acids, and/or expression vectors of the disclosure. Also disclosed is a method comprising contacting a cell with the polypeptide of the disclosure. Further aspects relate to a solid support comprising a polypeptide of the disclosure linked to or coated on the solid support.

In some aspects, (M)nGVADLIKKFE(X′)n′ (SEQ ID NO:47) or an amino acid sequence with at least 80% sequence identity to SEQ ID NO:47 is further defined as an actin-binding polypeptide, wherein X is any amino acid, n and n′ are each independently selected from 0 and 1, and X′ is equal to SISKEE. In some aspects, an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:47 is further defined as an actin-binding polypeptide, wherein X is any amino acid, n and n′ are each independently selected from 0 and 1, and X′ is equal to SISKEE. Method aspects of the disclosure relate to a method of making a cell comprising transferring an expression vector of the disclosure into a cell. Also described is a method of making a polypeptide comprising transferring the expression vector of claim 40 into a cell and incubating the cell under conditions that allow for the expression of the polypeptide. Further method aspects relate to a method for labeling actin in a cell comprising transferring an expression vector of the disclosure into the cell or contacting the cell with a polypeptide or the solid support of the disclosure.

In some aspect, the polypeptide comprises the amino acid sequence AXXIXXXAMGVADLIKKFESISKEE (SEQ ID NO:2) or an amino acid sequence with at least 80% sequence identity to SEQ ID NO:2. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 2. In some aspects, the polypeptide comprises the amino acid sequence AEEIDEAAMGVADLIKKFESISKEE (SEQ ID NO:3) or an amino acid sequence with at least 80% sequence identity to SEQ ID NO:3. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 3. In some aspects, the polypeptide comprises the amino acid sequence AEEIDEAAMEVADLIKKFESISKEE (SEQ ID NO:4) or an amino acid sequence with at least 80% sequence identity to SEQ ID NO:4. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 4. In some aspects, the polypeptide comprises the amino acid sequence AEEIDEAAMGAADLIKKFESISKEE (SEQ ID NO:5) or an amino acid sequence with at least 80% sequence identity to SEQ ID NO:5. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 5. In some aspects, the polypeptide comprises the amino acid sequence AEEIDEAAMAVADLIKKFESISKEE (SEQ ID NO:6) or an amino acid sequence with at least 80% sequence identity to SEQ ID NO:6. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 6. In some aspects, the polypeptide comprises the amino acid sequence AENIDEAAMGVADLIKKFESISKEE (SEQ ID NO:7) or an amino acid sequence with at least 80% sequence identity to SEQ ID NO:7. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 7. In some aspects, the polypeptide comprises the amino acid sequence AENIDEAAMEVADLIKKFESISKEE (SEQ ID NO:8) or an amino acid sequence with at least 80% sequence identity to SEQ ID NO:8. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 8. In some aspects, the polypeptide comprises the amino acid sequence AENIDEAAMGAADLIKKFESISKEE (SEQ ID NO:9) or an amino acid sequence with at least 80% sequence identity to SEQ ID NO:9. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 9. In some aspects, the polypeptide comprises the amino acid sequence AENIDEAAMAVADLIKKFESISKEE (SEQ ID NO:10) or an amino acid sequence with at least 80% sequence identity to SEQ ID NO: 10. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 10. In some aspects, the polypeptide comprises the amino acid sequence AEEIMGVADLIKKFESISKEE (SEQ ID NO: 11) or an amino acid sequence with at least 80% sequence identity to SEQ ID NO:11. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 11. In some aspects, the polypeptide comprises the amino acid sequence AENIMGVADLIKKFESISKEE (SEQ ID NO: 12) or an amino acid sequence with at least 80% sequence identity to SEQ ID NO: 12. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 12.

In some aspects, the polypeptide further comprises GEFLATTLERIEKNFVITDPRLPDNPIIFASDSFLQLTEYSREEILGRNCRFLQGPETDR ATVRKIRDAIDNQTEVTVQLINYTKSGKKFWNLFHLQPMRDQKGDVQYFIGVQLDG TEHVRDAAEREGVMLIKKT (SEQ ID NO:13) at the amino proximal position or an amino acid sequence with at least 70% sequence identity to SEQ ID NO: 13 at the amino proximal position. The polypeptide may further comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:13 at the amino proximal position. In some aspects, there is a salt bridge or the addition of one or more proline molecules between the polypeptide of SEQ ID NO: 13 and the polypeptide of SEQ ID NO:1. In some embodiments, the salt bridge or prolines are added to add a L-shaped kink between the polypeptide of SEQ ID NO:13 and the polypeptide of SEQ ID NO:1. The polypeptide may comprise, may comprise at least, or may comprise at most 1, 2, 3, 4, 5, 6, 7, 8, 9, 10, 11, 12, 13, 14, 15, 16, 17, 18, 19, 20, 21, 22, 23, 24, or 25 substitutions (or any derivable range therein) of a polypeptide described herein, such as a polypeptide of one of SEQ ID NOS: 1-38.

In some aspects, the polypeptide comprises a G128A substitution of SEQ ID NO: 13. In some aspects, the polypeptide comprises a 1132A substitution of SEQ ID NO:13. In some aspects, the polypeptide comprises a N14A substitution of SEQ ID NO:13. In some aspects, the polypeptide comprises a N14D substitution of SEQ ID NO:13. In some aspects, the polypeptide comprises a N14G substitution of SEQ ID NO:13. In some aspects, the polypeptide comprises a N14L substitution of SEQ ID NO:13. In some aspects, the polypeptide comprises a N14Q substitution of SEQ ID NO:13. In some aspects, the polypeptide comprises a N14S substitution of SEQ ID NO: 13. In some aspects, the polypeptide comprises a N14T substitution of SEQ ID NO:13. In some aspects, the polypeptide comprises a C50V substitution of SEQ ID NO:13. In some aspects, the polypeptide comprises a L53V substitution of SEQ ID NO:13. In some aspects, the polypeptide comprises a N92A substitution of SEQ ID NO: 13. In some aspects, the polypeptide comprises a F94L substitution of SEQ ID NO: 13. In some aspects, the polypeptide comprises a F94H substitution of SEQ ID NO: 13. In some aspects, the polypeptide comprises a Q113A substitution of SEQ ID NO: 13. In some aspects, the polypeptide comprises a Q113D substitution of SEQ ID NO:13. In some aspects, the polypeptide comprises a Q113H substitution of SEQ ID NO: 13. In some aspects, the polypeptide comprises a Q113L substitution of SEQ ID NO:13. In some aspects, the polypeptide comprises a N14A substitution and a Q113H substitution of SEQ ID NO:13. In some aspects, the polypeptide comprises a N14L substitution and a Q113A substitution of SEQ ID NO: 13. In some aspects, the polypeptide comprises a N14A substitution and a Q113A substitution of SEQ ID NO:13 . . . . In some aspects, the polypeptide comprises a F94C substitution of SEQ ID NO:13. In some aspects, the polypeptide comprises a C50V substitution and a F94C substitution of SEQ ID NO:13. In some aspects, the polypeptide comprises a C50V substitution and a Q113C substitution of SEQ ID NO:13. In some aspects, the polypeptide comprises a C50V substitution and a L53C substitution of SEQ ID NO: 13. In some aspects, the polypeptide comprises a N14T substation, a C50V substitution, and a Q113C substitution of SEQ ID NO: 13. In some aspects, the polypeptide comprises a N14G substation, a C50V substitution, and a Q113C substitution of SEQ ID NO:13. In some aspects, the polypeptide comprises the amino acid sequence of one of SEQ ID NOS: 15-46, or an amino acid sequence with at least 70% sequence identity to one of SEQ ID NOS: 15-46. The polypeptide may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS: 15-46. Methods of the disclosure may include the addition of imidazole. The concentration of imidazole may be at least, at most, or exactly 0.5, 1, 2, 3, 4, 5, or 6 mM, or any derivable range therein. The polypeptide may be one that has been contacted with imidazole. In some aspects, the methods include dehydration of the polypeptide. In some aspects, the polypeptide is one that has been dehydrated.

The polypeptide of the disclosure, including those discussed in the previous paragraph, may be further defined as a photoswitch. The term “photoswitch” refers to a molecule that is active upon exposure to light and inactive in the dark or has significantly decreased activity in the dark. In some aspects, the methods comprise or further comprise exposure of the polypeptide to light or to an excitation wavelength. In some aspects exposure to light or excitation wavelength comprises excitation of the polypeptide at a wavelength between 450-500 nm. In some aspects exposure to light comprises or the excitation wavelength of the polypeptide comprises a wavelength of at least, of at most, or of about 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, or 550 nm (or any derivable range therein). In some embodiments, the polypeptide has been or is being exposed to light. In some aspects, the methods comprise or further comprise exposure of the polypeptide to dark or to non-excitation wavelength of less than 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nm, or any derivable range therein or greater than 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 nm, or any derivable range therein. The decrease in activity in the dark or non-excitation wavelength may be, or may be at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease, or any derivable range therein. The activity may refer to a binding activity or a labeling activity. The polypeptide of the disclosure may be further defined as a polypeptide that binds to actin upon photo-excitation. In some aspects, the polypeptide has low affinity for actin in the dark (i.e. in the absence of photo-excitation). For example, the affinity for actin may be, may be at least, or may be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 μM (or any derivable range therein) in the dark. In some aspects, the affinity for the polypeptide in the dark or in a non-excitation wavelength may be about 10-500 μM. The polypeptide may be further defined as a polypeptide that binds to actin upon photo-excitation (i.e. upon exposure to light). In some aspects, the polypeptide has low affinity for actin in the dark. For example, the affinity for actin may be, may be at least, or may be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μM (or any derivable range therein) in light or at an excitation wavelength. In some aspects, the affinity for the polypeptide in the light or in an excitation wavelength may be about 2-10 μM.

The polypeptide may have a length of 250 amino acids or less. In some aspects, the polypeptide may have, have at least, or have at most 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 amino acids in lengths (or any derivable range therein).

In some aspects, the polypeptide is conjugated to at least one heterologous molecule. The heterologous molecule comprises a heterologous polypeptide. The heterologous molecule may be a fluorescent polypeptide such as GFP or a variant thereof. Other examples of heterologous molecules useful in the disclosure include, allophycocyanin, Brainbow, Cameleon (protein), Dronpa, Eos, Kaede, mCherry, phycoerythrin, red fluorescent protein, RoGFP, SmURFP, Synapto-pHluorin, and yellow fluorescent protein. The heterologous molecule may also be a non-peptidic molecule. The heterologous molecule may be a fluorescent labelling group such as fluorescein or rhodamine. The heterologous molecule may be coupled to the N-terminus of (or amino proximal to) the polypeptide or the heterologous molecule may be coupled to the C-terminus of (or carboxy proximal to) the polypeptide. A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise. The polypeptide further comprise one or more additional actin binding protein(s). The polypeptide may comprise or further comprise at least 1, 2, 3, 4, 5, 6, 7, 8, 9, or 10 actin binding proteins. The actin binding proteins may be repeated adjacent elements separated by 0, 1, 2, 3, 4, 5, 6, 10, 20, 30 or more amino acids or separated by other elements. In some aspects, at least one additional actin binding protein is at the N-terminus of the polypeptide. In some aspects, the additional actin binding protein(s) comprise LifeAct (SEQ ID NO:47), MGVADLIKKFESISKEE (SEQ ID NO:48), MEVADLIKKFESISKEE (SEQ ID NO:49), MGAADLIKKFESISKEE (SEQ ID NO:50), MAVADLIKKFESISKEE (SEQ ID NO:51), Utrophin, or F-tractin.

In the methods of the disclosure, the polypeptide may be provided in a composition at a certain concentration. The concentration may be at least, may be at most, or may be exactly 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg/ml or mg/ml (or any derivable range therein). In some aspects, the polypeptide is provided at a concentration of 5 μg/ml-10 mg/ml. The methods may comprise or further comprise contacting the polypeptide at a certain illumination intensity. The illumination intensity may be at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 Watts/cm², or any derivable range therein.

The cell of the disclosure may be further defined as a eukaryotic cell. The cell may be a fibroblast or muscle cell. The cell may also be a prokaryotic cell, such as a bacterial cell. In some aspects, the cell is a yeast cell, an insect cell, a mouse cell, an axolotl cell, a mammalian cell, or a human cell. In some aspects, the cell is a cardiomyocyte. The cell may also be a cell described herein. The cell may be a fixed cell or an un-fixed cell. In some aspects, the cell may be permeabilized. The cell may also be non-permeabilized. In some aspects, the expression vector is transferred into the cell. Fixing and permeabilizing cells generally locks them in place and makes it possible for larger molecules such as antibodies to access the interior of the cell for better targeting of the protein or condition you're interested in. But, fixed and permeabilized cells are dead, and one may lose the ability to look at dynamic biological processes. The term “fixing” with respect to cells refers to the addition of a cross-linking agent. Formaldehyde is the most commonly used fixative; it works by chemically bonding adjacent macromolecules, such as proteins, together. This process is known as crosslinking. Most available formaldehyde preparations are actually paraformaldehyde (PFA, polymeric formaldehyde) dissolved in water or a buffer. The free methanediols in the resulting solution are reactive with amine groups on proteins and other cellular structures that contain nitrogen. PFA also solubilizes some lipids in cellular membranes. PFA is commonly diluted to 3.7-5% v/v and is applied to cells for 10-15 minutes. In some aspects, the cell may be a non-dividing cell. In some aspects, the cell may be one that is difficult to transfect, such as a primary cell, a non-dividing cell, lymphoma cell, or embryonic stem cell, for example. The cell may also be a living cell. In some aspects, the cell is a cell in cell culture. In some aspects, the cell may be a live, non-fixed, cell. The cell may also be a live, non-fixed, non-permeabilized cell. The expression vector of the disclosure may be transferred into the cell by methods known in the art, including, but not limited to, transfection, electroporation, lipofection, and transduction.

The solid support of the disclosure may be a microscope slide, a chip, a surface for culturing cells, a bead, a microsphere, or a nanosphere. In some aspects, the solid support comprises a microscope slide. The polypeptide may be linked to the solid support covalently or the polypeptide may be linked to the solid support non-covalently. The polypeptide may be linked to the solid support through an antibody. For example, an antibody that binds to the polypeptide or portion thereof (such as a heterologous molecule linked to the polypeptide) may be coated or conjugated to the solid support. In some aspects, the polypeptide comprises a heterologous molecule and the antibody comprises an antibody that specifically recognizes the heterologous molecule. In some aspects, the heterologous molecule comprises a fluorescent protein.

Methods of the disclosure may comprise or further comprise isolating, purifying, imaging, or quantitating polypeptides expressed in the cell. In some aspects, the method further comprises isolating, purifying, imaging, or quantitating polypeptides expressed from the expression vector in the cell. In some aspects, the methods further comprising performing an assay described herein. Methods may comprise or further comprise transferring the expression vector into the cell and wherein the method further comprises quantitatively or qualitatively assaying for polypeptides expressed from the expression vector. In some aspects, the polypeptide comprises a fluorescent polypeptide or molecule. The methods may further comprise imaging and/or quantitating the fluorescent polypeptide or molecule. The methods may comprise or further comprise subtraction of the dark state or non-excitation state images from light state or excitation state images. The methods may further comprise performing an assay to detect or quantitate the polypeptide expressed from the expression vector. In some aspects, the methods comprise one or more of a western blot, an ELISA, immunofluorescence, or an immunoassay. The methods may comprise or further comprise an assay described herein. In some aspects, labeling comprises photo-excitation conditional labeling of actin. The term “photo-excitation conditional labeling” refers to labeling that occurs upon exposure to light at an excitation wavelength but does not occur in the dark or at a non-excitation wavelength or occurs to a lesser extent. The decrease in labeling in the dark may be, or may be at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease, or any derivable range therein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic of actin structures in the cell (1).

FIG. 2A-C: A) Schematic of LOV2 light absorption modified from PDB: 2VIA; B) UV/VIS spectra change upon photoexcitation; C) LOV2 absorbance at 488 nm over time (17).

FIG. 3A-B: LILAC reversibly recruits to S2 cell actin rings upon photoexcitation. A) Insect S2 cell expressing mCherry-LILAC pre/post excitation. B) Ratio of fluorescence inside/out of the cell over time. Black line is the exponential fit for the first recovery curve.

FIG. 4: Quantification of cell morphology effects of Lifeact. These effects are mitigated by alternatively expressing LILAC.

FIG. 5: Mutations to optimize LILAC dynamic range.

FIG. 6: Schematic of LILAC mechanism of action.

FIG. 7A-F: LILAC reversibly binds actin in S2 cells. a, Sequences of the LOV2 C-terminal Ja helix and Lifeact. The template was created by keeping hydrophobic residues (blue) which interact with the core of LOV2, and Lifeact was integrated to minimize disrupting those interactions. Grey: LOV2, turquoise: Ja helix, orange: key hydrophobic residues, black: Lifeact. b, Structural representations of LOV2 (left) and LILAC (right). Actin (blue) is shown in bound to Lifeact from PDB 7AD9. LOV2 residues that sterically clash with actin in the dark state are shown in dark red. The LILAC model was created by extending the Ja helix of AsLOV2 (PDB 2V0W) with residues of Lifeact in helical form. c, TIRF images of an S2 cell that is expressing LILAC taken before, during, and after excitation with blue light. d, Line scan of cell area highlighted in yellow in c before blue light excitation at 0 seconds and 1 to 60 seconds post excitation, where 0 μm is outside of the cell. Scale bar is 5 μm. e, Kymograph of the outer cell edge highlighted in yellow in (c), where the top is the inside of the cell, and the bottom is outside the cell. f, Actin labeling ratio of a repeatedly excited cell.

FIG. 8A-E: LILAC reduces the concentration dependent side effects of Lifeact. a, Cells at various intensities expressing Lifeact, LILAC, and LILACI539E. Cell circularity (b, P=1.57*10⁻⁴) and cell area (e, P=3.77*10⁻⁶) of Lifeact, LILAC, LILACI539E. Cell circularity (c), cell area (e) as a function of intensity with a linear regression fit to log-transformed data and shaded regions indicating the 95% confidence interval. For b, and d, P-values were determined initially using a one-way ANOVA test (n=27 for Lifeact, n=18 for LILAC, n=23 for LILACI539E). A post-hoc Dunn test was used to determine pairwise p-values (***=P≤0.001, ****=P≤0.0001).

FIG. 9A-D: LILAC highlights actin structures through background subtraction. TIRF image of an S2 cell expressing LILAC without (a) and with (c) pre-excitation image background subtraction to eliminate cytosolic background. Kymographs of the region in the box without (c) and with (d) pre-excitation background subtraction. e) Histograms of normalized pizel intensities in the outer ring of the cell with (CV=0.58) and without (CV=0.23) image subtraction.

FIG. 10A-B: Image processing of LILAC cells to obtain labeling and switching ratios An S2 cell expressing mCherry-LILAC before (a) and after (b) blue light excitation. The outer and inner rings of cells are hand traced in FIJI (yellow). Actin labeling ratio: ALR=Intensityₒᵤₜₑᵣ/Intensityᵢₙₙₑᵣ, actin switching ratio: ASR=ALRₚₒₛₜ/ALRₚᵣₑ.

FIG. 11A-D: Quantification of labeling and switching of LILAC. Max actin labeling ratio (a, R²=0.34, n=72) and switching ratio (b, R²=0.14, n=72) of LILAC expressing cells as a function of cell intensity. Maximum actin labeling (c, P=0.11) and switching ratios (d, P=9.66*10\hat{ }−15) of LILAC, Lifeact, and the light-mimic mutant, LILACI539E. The center line represents the median. Boxes span the 25^{th} and 75^{th} percentiles. Whiskers extend from the 25^{th} percentile −1.5× the interquartile range (IQR) and from the 75^{th} percentile +1.5× IQR. Data beyond the whiskers represent outliers and are plotted individually. For c and d, P-values were determined initially using a one-way ANOVA test (n=32 for Lifeact, n=18 for LILAC, n=23 for LILACI539E). A post-hoc Dunn test was used to determine pairwise p-values (**=P≤0.01, ***=P≤0.001, ****=P≤0.0001).

FIG. 12A-C: Dark state recovery time constants are cell specific and tunable. a, Min-max normalized actin labeling ratio traces of LILACT406A/T407A expressing cells with and without 1 mM imidazole. Cells were activated with blue light every 60 and 130 seconds respectively. b, First and second recovery time constants for individual cells expressing WT LILAC (R²=0.70), LILACT406A/T407A (R²=056), and LILACT406A/T407A with 1 mM imidazole (R²=0.19). Marginal kernel density estimates plotted along edges. d, Barplots of time constants of LILAC (n=34), LILACT406A/T407A (n=38), and LILACT406A/T407A with 1 mM imidazole (n=33) with error bars representing standard error (****=P≤0.0001).

FIG. 13: LILAC specifically labels U2OS actin structures post blue-light illumination. TIRF images of three U2OS cells expressing LILAC before and after excitation with blue light. The difference between pre and post excitation in shown on the right.

FIG. 14: LILAC specifically labels U2OS actin structures post blue-light illumination. TIRF images extracted from movies of a U20S cell expressing LILAC. In movie 1, the cell is not exposed to blue light between frames 1 and 2. In movie 2, the cell is excited by blue light between the frames. The difference between the two images in shown in the left column.

FIG. 15. LILAC labels actin patches in S2 cells. TIRF images of live S2 cells expressing mCherry-LILAC taken pre and post 488 nm (blue) laser excitation. Scale bar is 5 μm. Inset images are 3×.

FIG. 16. LILAC labels filopodia in landing S2 cells. TIRF images of live S2 cells expressing mCherry-LILAC taken pre and post 488 nm (blue) laser excitation. Cells are imaged as they land on the ConA coated coverslip. Scale bar is 5 μm. Inset images are 2×.

FIG. 17A-B. Phalloidin, Lifeact, and LILAC co-label actin structures in S2 cells. TIRF images of fixed S2 cells expressing (a) mCherry-Lifeact or (b) mCherry-LILAC stained with phalloidin. Cells were fixed under blue light. Pearson's correlation coefficients are displayed on the merge image. Scale bar is 5 μm.

FIG. 18A-B. Image processing methods with LILAC. (a) Post-excitation image of an S2 cell with pre-excitation image subtracted. (b) OLID image of an S2 cell. White pixels are correlated, black anticorrelated, and mid-grey are uncorrelated. Note the recruitment at the lamellipodium and the depletion in the cytosol. Also note how weaker lamellipodial actin signals are boosted compared to the back ground-subtracted image. The inventors used the first 50 frames of a movie (0.5 s/frame) where the excitation pulse arrives in frame 2 to generate the OLID image. For details, see methods. Scale bar, 5 μm.

FIG. 19A-B. Binding of Lifeact and LILAC to F-actin. A,b, SDS-PAGE of the protein of interest (P), either mCherry-LILACI539E (a) or mCherry-Lifeact (b), in the pellet after cosedimentation with 0.5 μM actin (A). Concentration of free protein added to each lane is labeled above in μM. c, Free protein plotted against bound protein in the pellet after cosedimentation to determined binding affinities of Lifeact (K_{d}=3.9 μM). Data was normalized to the maximum observed and fit to a hyperbola. The constitutively active form of LILAC was used because excitation of LILAC cannot be maintained in the dark ultracentrifuge.

FIG. 20A-B. LILAC excitation dynamics. a, Min-max normalized actin labeling ratio traces for cells initially in the dark, then activated with blue light continually through the end of the movie. b, Max switching ratios for cells excited for various lengths of time at 1% laser power, colored by cell.

FIG. 21A-E. Dark state recovery time constants are cell specific and tunable. a, First and second recovery time constants for individual cells expressing wild type (R²=0.70, n=34) and T406A/T407A (R²=0.56, n=23) LILAC. Grey line is X=Y. b, Kernel density estimations of the first time constants for wild type and T406A/T407A LILAC. c, Min-max normalized actin labeling ratio traces of LILAC (T406A/T407A) expressing cells with and without 1 mM imidazole. Cells were activated with blue light every 60 and 130 seconds respectively. d, First and second recovery time constants for individual cells expressing LILAC (T406A/T407A) with varying imidazole concentrations (R²=0.52, p=3.5e-24). Data with 0 and 1 mM imidazole were taken at different times with different starter cultures of S2 cells than all other concentrations, which were imaged on the same day.

FIG. 22A-F. LILAC constructs. Schematics of LILAC (a) and other vectors used. a, schematic of LILAC, including LOV2^{opt} (AsLOV2401-543 G528A/1532A/N538E). Vectors used for protein expression (b,c) and expression in S2 cells (d-f), where TEV is the TEV protease cleavage site.

FIG. 23. To ensure that the LILAC design is optimal, the inventors tested a second integration of Lifeact into the Jα helix. This design, known as LILAC2, inserts the Lifeact peptide after 1539, aligning A540 of LOV2 with A4 of Lifeact. They tested the switching of LILAC2 by imaging mCherry-LILAC2 in insect cells before and after excitation with blue light. S2 cells for lamellipodial rings, allowing for quantification of switching using the pre/post actin labeling ratio (intensity in lamellipodium/intensity in cell interior). LILAC2 does show increased switching in comparison to Lifeact (t-test 3.6 e-20). However, LILAC2 has a significantly lower switching ratio than LILAC (t-test p=1.8 e-4). Therefore, the inventors conclude that LILAC is the ideal integration of Lifeact with the Jα for maximal switching.

### DETAILED DESCRIPTION OF THE INVENTION

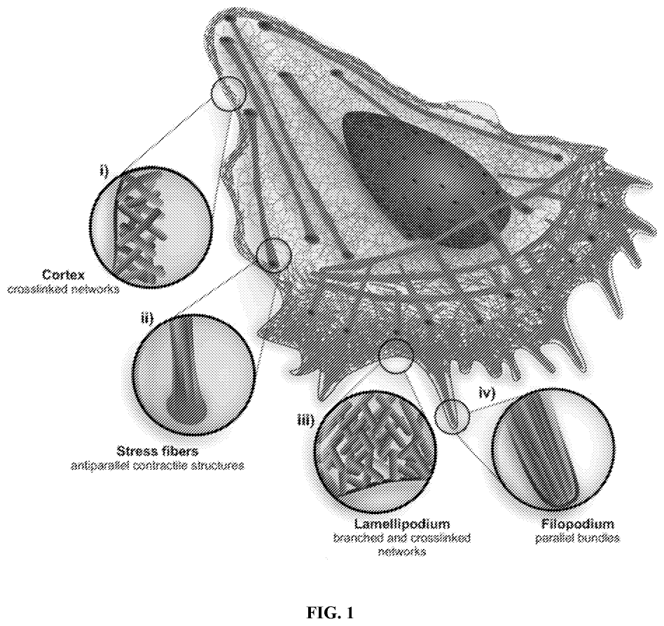

Actin filaments are the basic building block of a variety of cell structures, each integral for key cellular processes such as movement and cell division. The diversity of actin structures is a result of filaments forming branched and/or cross-linked filamentous networks, parallel filaments, and antiparallel filaments. A summary of some of the cellular structures built from these motifs is shown in FIG. 1. The formation of these distinct structures from the basic unit of an actin filament is made possible by the diverse array of actin binding proteins. These proteins can bundle, branch, push/pull, sever, cap filaments, and more (1). How these individual parts self organize in time and space to create subcellular structures and perform functions is an active research area.

A complete understanding of these complex networks requires in vitro study of purified proteins as well as their study in a cellular context. This is challenging, given most experiments can only be done using protein expression/labeling or addition of drugs to alter dynamics. However, these methods do not allow for modulation on the time scale which actin structures assemble and disassemble in cells. An elegant solution to this broad issue is a light-switchable actin binding protein, as light patterning allows for spatiotemporal control. Therefore, by attaching an actin binding protein of interest to this light activated label for actin (LILAC), one can investigate its effects on the cell at a subcellular level in real time. LILAC has potential use as a light-dependent label, crosslinker, recruiter, and more, allowing for answers to questions about cytoskeletal dynamics the inventors currently cannot determine

This disclosure describes a light-activated label for actin, or LILAC, by strategically integrating an ABP into a light-sensitive protein. The engineered proteins will be tested by expressing LILAC tagged with a fluorescent protein in cells and looking for reversible blue light recruitment of LILAC to filamentous actin. Additionally, the inventors will determine the dark and light binding affinities of LILAC to actin. The examples of the application also describe how one can investigate how ABPs segregate to different actin structures. In one approach, one could pattern actin to slides by illuminating LILAC coated slides to create various actin patterns, then after flowing various ABPs overtop, look for differential binding of various ABPs. It is proposed that ABPs use interfilament distance to sense actin structures. To test this hypothesis, the inventors will attach an N-terminal actin binding protein to LILAC to create a light activated cross-linker with various spectrin repeats to control interfilament distance and see if certain ABPs bind to filaments cross-linked at specific distances. The examples also describe methods to use LILAC to investigate the role of mechanotransduction in cardiomyocyte synchronicity. Some ABPs have been shown to bind at the same site as myosin heads to actin filaments, resulting in competitive binding. The inventors describe how one could illuminate cardiomyocytes expressing LILAC to determine the effect of cell stiffening on beating frequency, amplitude, and synchronicity. Together, these methods demonstrate the utility of LILAC for investigation of a wide array of questions about the actin cytoskeleton.

### I. Assays

The methods of the disclosure may include one or more biochemical assays. Such assays are known in the art. In some aspects, the methods include one or more of the following assays: 3D cell culture, cell fusion, cell suspension, an immunologic test, chromatin immunoprecipitation, cloned enzyme donor immunoassay, complement fixation test, counterimmunoelectrophoresis, direct fluorescent antibody, ELISA, epitope binning, epitope mapping, fluresence polarization immunoassay, immunoassay, immunocytochemistry, immunodiffusion, immunoelectrophoresis, immunofixation, immunofluorescence, immunohistochemistry, immunolabeling, immunoprecipitation, immunoradiometric assay, indirect immunoperoxidase assay, magnetic immunoassay, mass spectrometric immunoassay, MELISA, MeRIPseq, photopolymerization-based signal amplification, proximity ligation assay, radial immunodiffusion, radioallergosorbent test, radiobinding assay, radioimmunoassay, rapid antigen test, surround optical-fiber immunoassay, total complement activity, turbidimetric inhibition immunoassay, turbidimetry, widal test, ChIP sequencing, protein sequencing, horseradish peroxidase reaction, millon's reagent reaction, xanthoproteic reaction, electrophoresis, affinity electrophoresis, agarose gel electrophoresis, capillary electrochromatography, capillary electrophoresis, comet assay, dielectrophoresis, difference gel electrophoresis, discontinuous electrophoresis, DNA laddering, DNA separation by silica adsorption, electrical mobility, electroblotting, electrochromatography, electrophoresis, electrophoretic mobility shift assay, free-flow electrophoresis, gel electrophoresis, gel electrophoresis of nucleic acids, gel electrophoresis of proteins, immunoelectrophoresis, iontophoresis, isoelectric focusing, isotachophoresis, kinetic capillary electrophoresis, moving-boundary electrophoresis, process analytical chemistry, pulsed-field gel electrophoresis, QPNC-PAGE, SDS-PAGE, polyacrylamide gel electrophoresis, serum protein electrophoresis, temperature gradient gel electrophoresis, terminal restriction fragment length polymorphism, two-dimensional gel electrophoresis, microarray analysis, Affymetrix, AmpliChip, AmpliChip CYP450 Test, antibody microarray, biochip, cell-free protein array, chemical compound microarray, ChIP-on-chip, DNA microarray, FlexGen B.V., frozen tissue array, gene chip analysis, gene expression profiling, gene set enrichment analysis, methylation specific oligonucleotide microarray, microarray analysis techniques, peptide microarray, representation oligonucleotide microarray analysis, reverse phase protein lysate microarray, RNA immunoprecipitation chip, sequenom, SNP array, spectral genomics, suspension array technology, synthetic genetic array, tiling array, tissue microarray, virtual karyotype, polymerase chain reaction, protein imaging, electron microscope imaging, FMN-binding fluorescent protein imaging, green fluorescent protein imaging, PRIME (PRobe Incorporation Mediated by Enzymes) imaging, X-ray crystallography, affinity electrophoresis, bimolecular fluorescence complementation, chemically induced dimerization, chromatin immunoprecipitation, complementarity plot, cross-link, dual-polarization interferometry, flow-induced dispersion analysis, focal molography, Förster resonance energy transfer, glycan-protein interactions, immunoprecipitation, intensity fading MALDI mass spectrometry, methods to investigate protein-protein interactions, multi-parametric surface plasmon resonance, phage display, photo-reactive amino acid analog, single colour reflectometry, SPINE (molecular biology), split TEV, stable isotope labeling by amino acids in cell culture, surface plasmon resonance, tandem affinity purification, two-hybrid screening, western blotting, co-immunoprecipitation, label transfer, phage display, tandem affinity purification, chemical cross-linking, MALDI mass spectrometry, quantitative immunoprecipitation combined with knock-down, proximity ligation assay, surface plasmon resonance, dual polarization interferometry, static light scattering, dynamic light scattering, fluorescence polarization, fluorescence resonance energy transfer, bio-layer interferometry, nuclear magnetic resonance, isothermal titration calorimetry, microscale thermophoresis, and single color reflectometry. The methods of the disclosure may also exclude one or more of the assays described herein and those recited above.

### II. Proteins

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS: 1-54.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS: 1-54.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs: 1-54.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOS: 1-54 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 1-54.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS: 1-54 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS: 1-54.

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS: 1-54 (or any derivable range therein) and may be a substitution with any amino acid or may be a substitution with an alanine, arginine, asparagine, aspartic acid, cysteine, glutamine, glutamic acid, glycine, histidine, isoleucine, leusine, lysine, methionine, phenylilacnine, proline, serine, threonine, tryptophan, tyrosine, or valine.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylilacnine to tyrosine, leucine or methionine; serine to threonine; threonine to serine; tryptophan to tyrosine; tyrosine to tryptophan or phenylilacnine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylilacnine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylilacnine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### III. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### IV. Obtaining Encoded Polypeptide Embodiments

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### V. Formulations and Culture of the Cells

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine) . . . . In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or futher comprises amino acids, monosaccharides, inorganic ions. In some embodiments, the amino acids comprise arginine, cystine, isoleucine, leucine, lysine, methionine, glutamine, phenylilacnine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylilacnine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

### VI. Kits

Certain aspects of the present disclosure also concern kits containing nucleic acids, vectors, or cells of the disclosure. The kits may be used to implement the methods of the disclosure. In some embodiments, kits can be used to evaluate or facilitate binding of the polypeptides to actin. Kits may also include reagents for performing an assay described herein. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more nucleic acid probes, primers, or synthetic RNA molecules, or any value or range and combination derivable therein. In certain embodiments, the kits may comprise materials for analyzing cell morphology and/or phenotype, such as histology slides and reagents, histological stains, alcohol, buffers, tissue embedding mediums, paraffin, formaldehyde, and tissue dehydrant.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, polypeptide or polynucleotide detecting agents of the disclosure for drug discovery are contemplated.

In certain aspects, negative and/or positive control agents are included in some kit embodiments. The control molecules can be used to verify transfection efficiency and/or control for transfection-induced changes in cells.

Embodiments of the disclosure include kits for analysis of a pathological sample by assessing a nucleic acid or polypeptide profile for a sample comprising, in suitable container means, two or more RNA probes or primers for detecting expressed polynucleotides. Furthermore, the probes or primers may be labeled. Labels are known in the art and also described herein. In some embodiments, the kit can further comprise reagents for labeling probes, nucleic acids, and/or detecting agents. The kit may also include labeling reagents, including at least one of amine-modified nucleotide, poly (A) polymerase, and poly (A) polymerase buffer. Labeling reagents can include an amine-reactive dye. Kits can comprise any one or more of the following materials: enzymes, reaction tubes, buffers, detergent, primers, probes, antibodies. In some embodiments, these kits include the needed apparatus for performing RNA extraction, RT-PCR, and gel electrophoresis. Instructions for performing the assays can also be included in the kits.

The kits may further comprise instructions for using the kit for assessing expression, means for converting the expression data into expression values and/or means for analyzing the expression values to generate ligand/receptor interaction data.

Kits may comprise a container with a label. Suitable containers include, for example, bottles, vials, and test tubes. The containers may be formed from a variety of materials such as glass or plastic. The container may hold a composition which includes a probe that is useful for the methods of the disclosure. The kit may comprise the container described above and one or more other containers comprising materials desirable from a commercial and user standpoint, including buffers, diluents, filters, needles, syringes, and package inserts with instructions for use.

### VII. Sequences

| | | SEQ |
|---|---|---|
| | | ID |
| Description | Sequence | NO: |
| LILAC - G128A; | GEFLATTLERIEKNFVITDPRLPDNPIIFASDSFLQLTEYSREE | 15 |
| I132A; N138E | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REAVMLAKKTAEEIDEAAMGVADLIKKFESISKEE | |
| LILAC - N138E | GEFLATTLERIEKNFVITDPRLPDNPIIFASDSFLQLTEYSREE | 16 |
| | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIDEAAMGVADLIKKFESISKEE | |
| LILAC - G128A; | GEFLATTLERIEKNFVITDPRLPDNPIIFASDSFLQLTEYSREE | 17 |
| I132A; N138E; | ILGRNCRFLQGPETDRATVRKIRDAIDNQTEVTVQLINYTKS | |
| G145E | GKKFWNLFHLQPMRDQKGDVQYFIGVQLDGTEHVRDAAE | |
| | REAVMLAKKTAEEIDEAAMEVADLIKKFESISKEE | |
| LILAC - N138E; | GEFLATTLERIEKNFVITDPRLPDNPIIFASDSFLQLTEYSREE | 18 |
| G145E | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIDEAAMEVADLIKKFESISKEE | |
| LILAC - G128A; | GEFLATTLERIEKNFVITDPRLPDNPIIFASDSFLQLTEYSREE | 19 |
| I132A; N138E; | ILGRNCRFLQGPETDRATVRKIRDAIDNQTEVTVQLINYTKS | |
| V146A | GKKFWNLFHLQPMRDQKGDVQYFIGVQLDGTEHVRDAAE | |
| | REAVMLAKKTAEEIDEAAMGAADLIKKFESISKEE | |
| LILAC - N138E; | GEFLATTLERIEKNFVITDPRLPDNPIIFASDSFLQLTEYSREE | 20 |
| V146A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIDEAAMGAADLIKKFESISKEE | |
| LILAC - G128A; | GEFLATTLERIEKNFVITDPRLPDNPIIFASDSFLQLTEYSREE | 21 |
| I132A; N138E; | ILGRNCRFLQGPETDRATVRKIRDAIDNQTEVTVQLINYTKS | |
| G145A | GKKFWNLFHLQPMRDQKGDVQYFIGVQLDGTEHVRDAAE | |
| | REAVMLAKKTAEEIDEAAMAVADLIKKFESISKEE | |
| LILAC - N138E; | GEFLATTLERIEKNFVITDPRLPDNPIIFASDSFLQLTEYSREE | 22 |
| G145A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIDEAAMAVADLIKKFESISKEE | |
| LILAC - G128A; | GEFLATTLERIEKNFVITDPRLPDNPIIFASDSFLQLTEYSREE | 23 |
| I132A; | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REAVMLAKKTAENIDEAAMGVADLIKKFESISKEE | |
| LILAC - | GEFLATTLERIEKNFVITDPRLPDNPIIFASDSFLQLTEYSREE | 24 |
| | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIDEAAMGVADLIKKFESISKEE | |
| LILAC - G128A; | GEFLATTLERIEKNFVITDPRLPDNPIIFASDSFLQLTEYSREE | 25 |
| I132A; G145E | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REAVMLAKKTAENIDEAAMEVADLIKKFESISKEE | |
| LILAC - G145E | GEFLATTLERIEKNFVITDPRLPDNPIIFASDSFLQLTEYSREE | 26 |
| | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIDEAAMEVADLIKKFESISKEE | |
| LILAC - G128A; | GEFLATTLERIEKNFVITDPRLPDNPIIFASDSFLQLTEYSREE | 27 |
| I132A; V146A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REAVMLAKKTAENIDEAAMGAADLIKKFESISKEE | |
| LILAC - V146A | GEFLATTLERIEKNFVITDPRLPDNPIIFASDSFLQLTEYSREE | 28 |
| | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIDEAAMGAADLIKKFESISKEE | |
| LILAC - G128A; | GEFLATTLERIEKNFVITDPRLPDNPIIFASDSFLQLTEYSREE | 29 |
| I132A; G145A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REAVMLAKKTAENIDEAAMAVADLIKKFESISKEE | |
| LILAC - G145A | GEFLATTLERIEKNFVITDPRLPDNPIIFASDSFLQLTEYSREE | 30 |
| | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIDEAAMAVADLIKKFESISKEE | |
| LILAC2 - | GEFLATTLERIEKNFVITDPRLPDNPIIFASDSFLQLTEYSREE | 31 |
| G128A; I132A; | ILGRNCRFLQGPETDRATVRKIRDAIDNQTEVTVQLINYTKS | |
| N138E | GKKFWNLFHLQPMRDQKGDVQYFIGVQLDGTEHVRDAAE | |
| | REAVMLAKKTAEEIMGVADLIKKFESISKEE | |
| LILAC2 - | GEFLATTLERIEKNFVITDPRLPDNPIIFASDSFLQLTEYSREE | 32 |
| G128A; I132A; | ILGRNCRFLQGPETDRATVRKIRDAIDNQTEVTVQLINYTKS | |
| N138E; G141E | GKKFWNLFHLQPMRDQKGDVQYFIGVQLDGTEHVRDAAE | |
| | REAVMLAKKTAEEIMEVADLIKKFESISKEE | |
| LILAC2 - | GEFLATTLERIEKNFVITDPRLPDNPIIFASDSFLQLTEYSREE | 33 |
| G128A; I132A; | ILGRNCRFLQGPETDRATVRKIRDAIDNQTEVTVQLINYTKS | |
| N138E; V142A | GKKFWNLFHLQPMRDQKGDVQYFIGVQLDGTEHVRDAAE | |
| | REAVMLAKKTAEEIMGAADLIKKFESISKEE | |
| LILAC2 - | GEFLATTLERIEKNFVITDPRLPDNPIIFASDSFLQLTEYSREE | 34 |
| G128A; I132A; | ILGRNCRFLQGPETDRATVRKIRDAIDNQTEVTVQLINYTKS | |
| N138E; G141A | GKKFWNLFHLQPMRDQKGDVQYFIGVQLDGTEHVRDAAE | |
| | REAVMLAKKTAEEIMAVADLIKKFESISKEE | |
| LILAC2 - | GEFLATTLERIEKNFVITDPRLPDNPIIFASDSFLQLTEYSREE | 35 |
| G128A; I132A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REAVMLAKKTAENIMGVADLIKKFESISKEE | |
| LILAC2 - | GEFLATTLERIEKNFVITDPRLPDNPIIFASDSFLQLTEYSREE | 36 |
| G128A; I132A; | ILGRNCRFLQGPETDRATVRKIRDAIDNQTEVTVQLINYTKS | |
| G141E | GKKFWNLFHLQPMRDQKGDVQYFIGVQLDGTEHVRDAAE | |
| | REAVMLAKKTAENIMEVADLIKKFESISKEE | |
| LILAC2 - | GEFLATTLERIEKNFVITDPRLPDNPIIFASDSFLQLTEYSREE | 37 |
| G128A; I132A; | ILGRNCRFLQGPETDRATVRKIRDAIDNQTEVTVQLINYTKS | |
| V142A | GKKFWNLFHLQPMRDQKGDVQYFIGVQLDGTEHVRDAAE | |
| | REAVMLAKKTAENIMGAADLIKKFESISKEE | |
| LILAC2 - | GEFLATTLERIEKNFVITDPRLPDNPIIFASDSFLQLTEYSREE | 38 |
| G128A; I132A; | ILGRNCRFLQGPETDRATVRKIRDAIDNQTEVTVQLINYTKS | |
| G141A | GKKFWNLFHLQPMRDQKGDVQYFIGVQLDGTEHVRDAAE | |
| | REAVMLAKKTAENIMAVADLIKKFESISKEE | |
| LILAC2 - | GEFLATTLERIEKNFVITDPRLPDNPIIFASDSFLQLTEYSREE | 39 |
| N138E | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIMGVADLIKKFESISKEE | |
| LILAC2 - | GEFLATTLERIEKNFVITDPRLPDNPIIFASDSFLQLTEYSREE | 40 |
| N138E; G141E | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIMEVADLIKKFESISKEE | |
| LILAC2 - | GEFLATTLERIEKNFVITDPRLPDNPIIFASDSFLQLTEYSREE | 41 |
| N138E; V142A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIMGAADLIKKFESISKEE | |
| LILAC2 - | GEFLATTLERIEKNFVITDPRLPDNPIIFASDSFLQLTEYSREE | 42 |
| N138E; G141A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAEEIMAVADLIKKFESISKEE | |
| LILAC2 - | GEFLATTLERIEKNFVITDPRLPDNPIIFASDSFLQLTEYSREE | 43 |
| | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIMGVADLIKKFESISKEE | |
| LILAC2 - | GEFLATTLERIEKNFVITDPRLPDNPIIFASDSFLQLTEYSREE | 44 |
| G141E | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIMEVADLIKKFESISKEE | |
| LILAC2 - | GEFLATTLERIEKNFVITDPRLPDNPIIFASDSFLQLTEYSREE | 45 |
| V142A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIMGAADLIKKFESISKEE | |
| LILAC2 - | GEFLATTLERIEKNFVITDPRLPDNPIIFASDSFLQLTEYSREE | 46 |
| G141A | ILGRNCRFLQGPETDRATVRKIRDAIDNQTEVTVQLINYTKS | |
| | GKKFWNLFHLQPMRDQKGDVQYFIGVQLDGTEHVRDAAE | |
| | REGVMLIKKTAENIMAVADLIKKFESISKEE | |

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Development of Optogenetic Polypeptides

The cytoskeleton is a core component of the cell, responsible for cell structure and movement. Imaging and spatiotemporal control of the cytoskeleton are necessary to understand how the components of the cytoskeleton self assemble to perform various functions. The inventors sought to design a light-switchable actin binding protein and use this tool in multiple contexts including reducing the side effects of imaging reagents, patterning and cross-linking actin to determine how proteins recognize actin structures, and finally, modulating cardiomyocyte beating to understand how movement affects cell-cell communication

The actin cytoskeleton is at the heart of a range of cellular processes, from cellular adhesion, shape, and movement, to endo- and exocytosis and cytokinesis. Therefore, dependable actin visualization is vital for various fields of biological research. Phalloidin, a toxin from the death cap mushroom, is the gold standard for actin filament visualization. However, its high specificity for actin filaments is the result of its binding at the interface between two subunits (26). Stabilization of filaments, as well as membrane impermeability, limit phalloidin's use to fixed-staining. For live-cell imaging, direct labeling of actin monomers by a fluorescent protein (FP) is possible but gives background signal from FP-actin monomers and has been linked to alterations in actin structure and dynamics (27). As an alternative, actin binding domains such as Utrophin, F-Tractin, and actin chromobodies can be tagged with an FP (28). The downsides to these approaches include non-uniform labeling of f-actin and alteration of actin dynamics. For one actin binding domain from yeast, actin binding protein 140 (ABP140) it was shown that the first 17 amino acids, renamed Lifeact, were sufficient to bind actin (7). The short sequence was shown to be conserved in relatives of Saccharomyces cerevisiae but is not found in other organisms. Since its discovery, Lifeact-FP has been used in over 1,800 studies, at unspecified concentrations. Courtemanche et al. found that Lifeact has dose-dependent effects on actin dynamics in fission yeast (29). Increased Lifeact concentration led to slowed actin patch formation and disassembly, as well as contractile ring assembly and restriction. It is suggested that this is due to Lifeact perturbing cofilin-actin binding (29). Lifeact also alters cellular morphology and biophysical properties as well as stress fiber dynamics and structure in a dose dependent manner in human stem cells (30). A new structural study found that Lifeact binds to actin at the D-loop, overlapping with the binding interface of both cofilin and myosin to actin (22,23). These findings together suggest that competitive binding of Lifeact with cofilin and myosin leads to unwanted side effects when used as a label. Because of these multiple unwanted effects of Lifeact, the inventors sought to make a modified version that reduced or limited these effects so that natural processes could more accurately be studied.

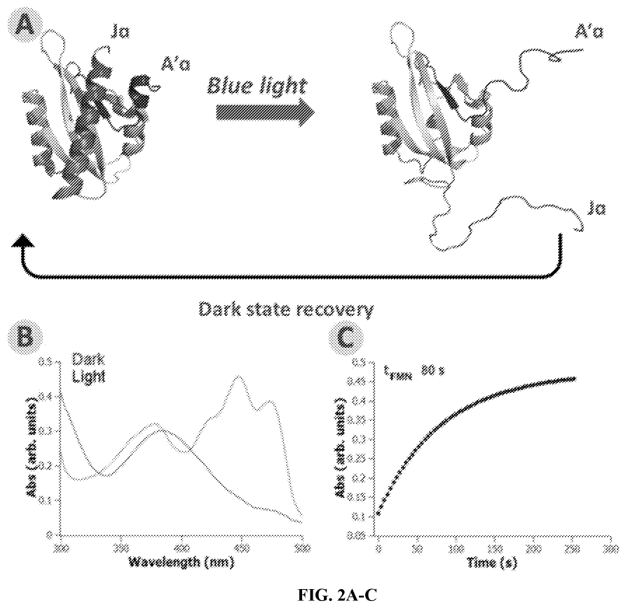

Avena sativa LOV2 (LOV2) is the second LOV (light oxygen voltage) domain from phototropinI in oats (4-6). It is involved in the phototropism response in plants, which includes chloroplast arrangement and growth towards light, and noncovalently binds a flavin mononucleotide (31,32). In response to blue light absorption, the flavin chromophore forms an adduct with C450, resulting in conformational changes within the core (6). This disrupts the 7 amino acid N-terminal A′a helix and 24 amino acid C-terminal Jα helix, causing them to unfold (33). (FIG. 2A). The deprotonation of the N5 atom of flavin returns the chromophore to its dark state leading to refolding of the A′a and Jα helices. This dark state recovery has a time constant of 80 sec for the wild type protein (FIG. 2B-C). This time constant is tunable over several orders of magnitude through mutations and buffer conditions (17,34). NMR showed 600-fold switching leading to a free energy difference of ˜3.8 kcal/mol (38).

### A. Approach

The inventors first sought to design a light-triggered reversible actin binding protein to reduce its side effects in cells. Although Lifeact has been shown to alter actin dynamics (29), cellular morphology (30), and ABP binding (22,23), it is still widely used as a label for actin filaments. To solve this problem, the inventors will, engineer and characterize a light activated Lifeact so that cells can be grown in the dark with a low effective concentration of Lifeact to decrease concentration dependent side effects.

Lifeact is a good candidate for an actin binding peptide to transform into an optogenetic tool for two reasons: it is widely used despite its concentration dependent effects, and it is a short helical peptide. In accordance with the previous design of LOV2 caged proteins, the inventors will first determine the optimal fusion of LOV2 to Lifeact. To do this, they first determined which amino acids of the Jα helix formed contacts with the core of the protein and created a template based off of the spacing between those required amino acids. Next, the inventors aligned the Lifeact sequence with this template, attempting to only mutate contact-forming amino acids to other hydrophobic residues. This resulted in candidate sequences of LILAC.

Preliminary data shows that LILAC1 binds actin in a light-dependent manner. The inventors then transfected insect macrophage-like S2 cells with mCherry tagged LILAC and imaged them in the dark (just 561 nm to excite mCherry), then in the light (561 nm+488 nm to excite LOV2 as well) to test for recruitment to actin filaments. Insect cells were used due to their widespread use and easily detectable actin rings on the outer edge of the cell.

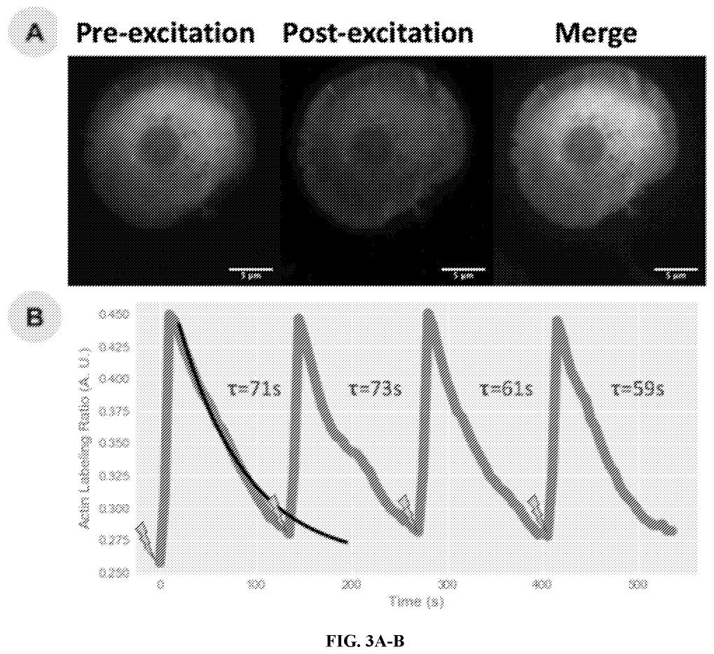

The inventors varied plasmid uptake to create a distribution of LILAC concentrations in the cells. This distribution is beneficial as too little expression leads to low signal whereas high expression could lead to binding in the dark, as some uncaged LILAC exists even in the dark (>1.6%), and is able to saturate actin filaments. Preliminary TIRF data shows LILAC is recruited to the outer ring of the cell post excitation (FIG. 3A). To quantify how well the actin is labeled, the inventors took a ratio of the average fluorescence in the outer ring of the cell and the inner area. FIG. 3B shows that LILAC binds reversibly to the actin-rich outer ring of the cell after blue light exposure and exponentially recovers to pre-excitement levels with time constants similar to the characteristic 80 second recovery time of LOV2.

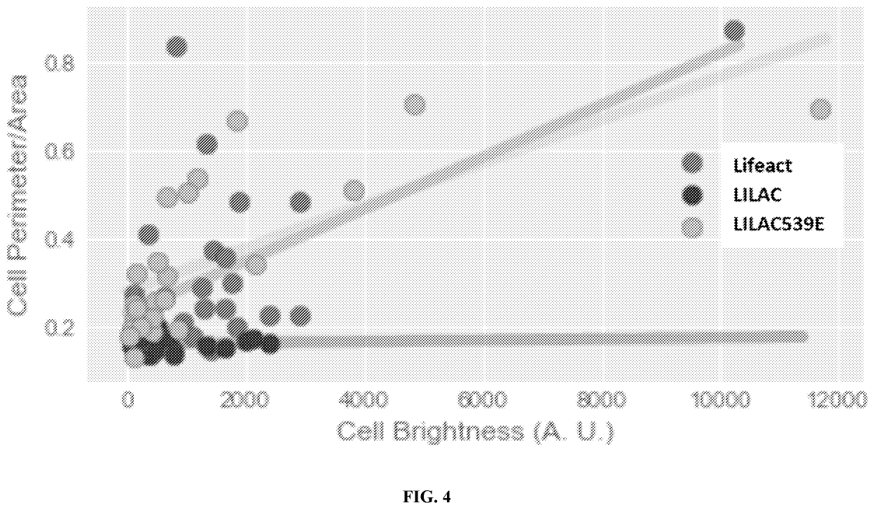

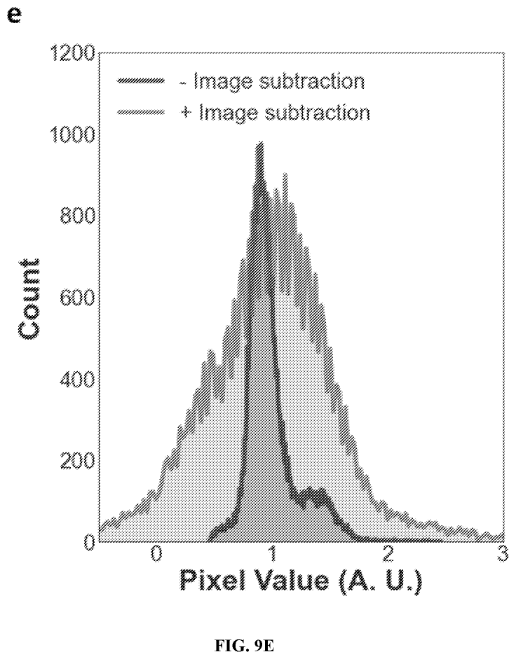

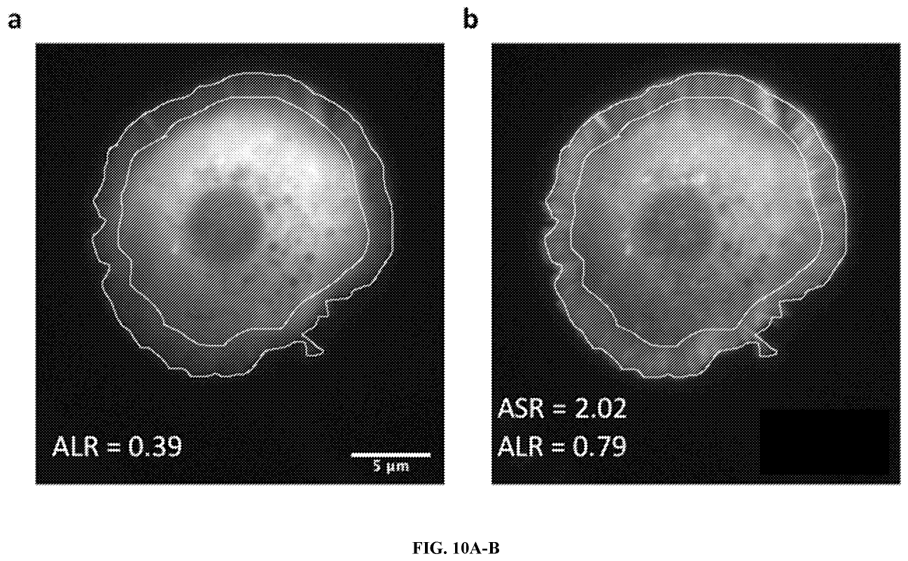

The inventors sought to determine whether LILAC reduces concentration dependent side effects of Lifeact. Lifeact has been shown to disrupt various cells, but its concentration dependent side effects have not been researched in S2 cells. The inventors transfected S2 cells with mCherry tagged Lifeact and found that brighter cells (more highly expressing) had thick, long actin structures and protrusions unlike LILAC expressing cells of similar brightness (FIG. 9A-top row). To quantify this “spikiness” the inventors used the ratio of the perimeter to the area of each cell. For round cells, this should be twice the inverse radius of the cell (2piR/piR\hat{ }2=2/R), but will be larger for spikier cells. Thus, FIG. 4 shows that LILAC mitigates the concentration dependent side effects of Lifeact on S2 cells. Additionally, the constitutively unfolded 539E mutation recapitulates the Lifeact phenotype, suggesting that LILAC binds actin with a similar affinity to Lifeact.

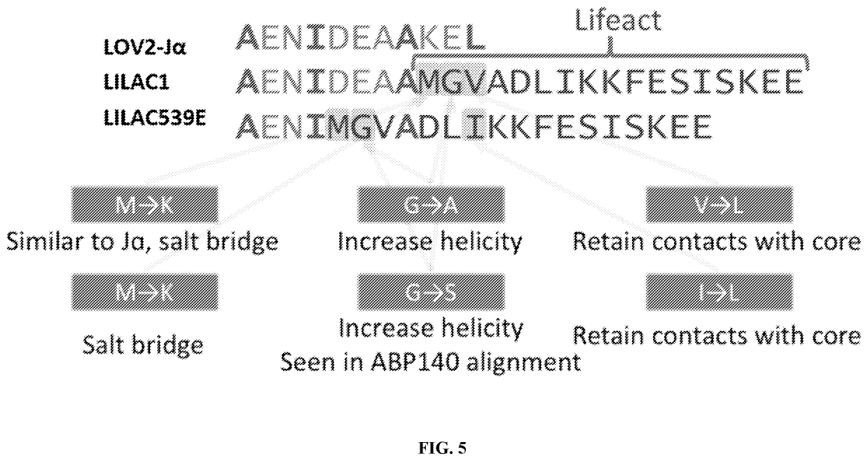

Ideally, optogenetic tools have little “leakage”, or dark state binding, and high dynamic range, or switching ratio, between dark and light state binding. To maximize these parameters, the inventors planned the engineering of different polypeptides with substitutions (FIG. 5). They hypothesize that substitution of methionine of Lifeact to lysine may create EK or DK salt bridges that stabilize the helix and strengthen caging. Glycine is a flexible amino acid, making it entropically favorable to be unfolded compared to other residue types. They hypothesized that substituting the glycine to alanine or serine will stabilize the helix. They also hypothesized that substituting the valine in LILAC1 and isoleucine in LILAC2 to leucine may maintain the contacts between the helix and the core. These engineered polypeptides may have the ability to impede actin binding by altering the Lifeact sequence, making it necessary to determine the light state binding constant. To determine binding constants in the light and dark, the inventors will use cosedimentation of actin with 6×His-mCherry-LILAC in E. coli. Cosedimentation of actin filaments with ABPs is canonically done at greater than 100,000×g (39,40). Inserting blue lights into an ultracentrifuge spinning at these speeds is infeasible, so the inventors will instead perform the pull down assay with bundled actin, which can be pelleted at 10,000×g in a small table top centrifuge (41,42). The inventors have already tested that blue LED lights can be safely inserted into these centrifuges at these speeds. Gel analysis of LILAC contained in the pellet versus the supernatant in light/dark will give the binding constants to assess which LILAC design has the largest dynamic range while maintaining minimal dark state leakage. This will be compared to the binding constant of 6×His-mCherry-Lifeact to bundled actin filaments.

The inventors also seek to use LILAC as a tool for actin recruitment and crosslinking. The spatial and temporal organization of the actin cytoskeleton is fundamental to cells, yet there is currently no understanding of the underlying principles of how these filaments self organize (46-48). Additionally, it is currently unknown exactly how ABPs recognize specific actin networks, despite many ABPs being well characterized biochemically. The inventors plan to address these questions by spatiotemporally recruiting actin with LILAC and by using LILAC to cross link actin filaments with various interfilament distances to see if these bundlers cosegregate.

The inventors plan to pattern LILAC with temporal resolution. Current tool are limited to spatial organization of actin filaments in vitro via micropatterning glass slides with actin filament nucleation factors (49-52). By recruiting actin filaments with LILAC, the inventors will elucidate the spatial and geometric constraints that affect filament organization. First, the inventors will pattern actin filaments on flow cells created on glass coverslips. An important consideration is the choice of fluorescent labels, since one need to ensure that visualizing actin does not also excite LOV at 488 nm. Therefore the inventors plan to use either red or infrared phalloidin, which does not affect Lifeact binding (7,23), to stain the actin filaments. Phalloidin is also commercially available and resistant to photobleaching. The inventors will fix FP-LILAC to slides by attaching anti-FP antibodies to nitrocellulose or plasma coated coverslips. To reduce non-specific binding of filaments to the coated coverslips, next, the inventors will flow BSA over the slides, wait a few minutes, then flow stained actin filaments. Lastly, the inventors will use a digital mirror device (DMD) to illuminate the coverslip with patterned blue light, activating LILAC in the illuminated areas and recruiting actin, and record the image using a TIRF microscope. The TIRF microscope takes advantage of evanescent field's limited penetration to illuminate a thin layer above the coverslip, allowing for more precise visualization of recruitment of actin to LILAC coated coverslips. If successful, more red actin filaments will reside in the area illuminated by blue light than in the control area. From these videos, the inventors will extract time constants for association and dissociation of actin filaments from fixed LILAC. The inventors will also change parameters such as LILAC concentration, illumination intensity and filament size to see how these affect association and dissociation time constants.

An advantage of using this optogenetic tool is the ability to spatiotemporally pattern actin filaments. One desirable example is translating parallel bars of light. This pattern would mimic the creation of actin filaments in lamellipodia at cell edges. For studying round cells, another pattern would be outward moving concentric rings. The effects of speed and thickness of the rings/bars can be studied. Previous research has used patterning of actin nucleation factors to investigate how network architecture determines disassembly of filaments by cofilin (49). The inventors plan to expand on this work by examining the interactions between ABPs and actin networks as they are formed, instead of using static, pre-formed structures. To investigate how actin binding proteins affect actin network as they are formed, the inventors will form patterned actin networks in the presence of bundling proteins such as fascin and alpha-actinin, and branching proteins, such as the Arp2/3 complex, to more accurately mimic the formation of these networks in cells. The inventors predict that pre-bundled filaments will recruit to illuminated areas faster due to the increased number of possible binding sites on the filament bundles. To visualize the creation of branched actin networks, the inventors will coat the coverslips in both LILAC and the Arp2/3 complex. Then, when actin is recruited to LILAC spatiotemporally, it will increase local concentration of Arp2/3 to increase filament branching.

The inventors also sought to determine if ABPs detect interfilament distance. The inventors will use a Lifeact-Spectrin (X)-LILAC or “LAAX” chimera as a light-sensitive cross linking protein. In cells, there are two main categories of bundled filaments: tightly packed parallel networks containing fimbrin, espin and fascin that make up filopodia and microvilli, and widely spaced, mixed polarity networks crosslinked with alpha-actinin that make up stress fibers, sarcomeres, and cytokinetic rings (1, 41-48). Despite the importance of crosslinkers to actin network structure and stiffness, the field lacks a spatiotemporally controllable actin crosslinker. Additionally, with this tool, the inventors will determine if ABPs use interfilament distance to differentiate actin structures. Winkelman et. al. found that compact bundlers fimbrin, fascin, and espin cosegregate from alpha-actinin bundled filaments, suggesting that these proteins recognize interfilament distance (42). To test this hypothesis, the inventors will image actin filaments as they crosslink with varying interfilament distances and observe if fascin/alpha-actinin recruit to tightly or loosely cross-linked filaments. The first step in creating the light-activated actin LAAX crosslinker will be to add an N-terminal Lifeact peptide to LILAC to anchor one side to a filament.

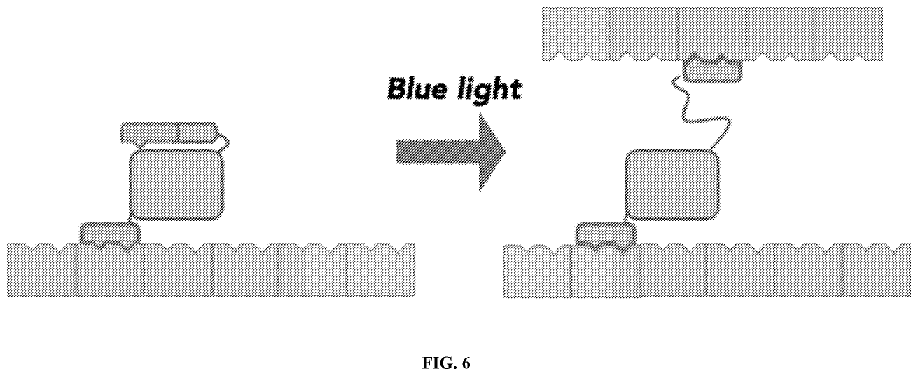

Then upon illumination, the second Lifeact peptide will uncage, making LAAX able to bind two filaments (FIG. 6). Based on the dimensions of AsLOV2, this should provide an interfilament distance of ˜6 nm, which is similar to the interfilament distance of fascin bundles. To increase the interfilament distance to the nearly 35 nm of alpha-actinin bundles, the inventors will add spectrin repeats as spacers (Lifeact-SpectrinX-LILAC), which are ˜5 nm long. If fascin and alpha-actinin recognize interfilament distance, fascin will preferentially colocalize to light activated areas crosslinked with LAAX without spectrin repeats, while alpha-actinin will recruit to bundles made with LAAX with at least 6 spectrin repeats.

The inventors also sought to alter beating dynamics of cardiomyocytes through competitive binding of LILAC and myosin. The coordination of cardiomyocyte contraction in space and time is critical for successful beating of the heart. Therefore, optogenetic tools are an optimal method as one can express them in living cells and control them noninvasively with high spatiotemporal resolution. Two main causes of heart arrhythmia are channelopathies, which are genetic defects in cardiac ion channels, and cardiac infarction, or heart attack. Previous use of optogenetics to investigate channelopathies used opsin-based optogenetics (56-60). These light-sensitive ion channels are useful for examining electrical activation and communication between cells, and have been extensively used as a less invasive alternative to electrical stimulation. Unlike previous cardiac optogenetics, The inventors plan to use LILAC to alter the physical, not electrical, properties of beating cardiomyocytes. This will allow them to understand how cardiac mechanotransduction affects cardiomyocyte beating.

During myocardial infarction, reduced blood flow decreases ATP in myocytes (61-63). Without ATP, cells cannot repolarize and provide energy for the myosin power stroke. The influence of electrochemical signaling has been extensively studied in myocytes, while less focus has been on the influence of cell stiffening. Deformation triggers mechanotransduction signaling pathways including stretch activated ion channels and changes in chemical gradients (62-65). Mechanotransduction allows cells to modulate cell stiffness in response to deformation. Titin and collagen in the extracellular matrix drive the passive biomechanical properties of the heart, while actin and myosin provide active contractile forces (62,63). Therefore, spatiotemporal reduction of acto-myosin contraction will elucidate how infarction-induced stiffening alters beating dynamics.

To reduce the beating of HL-1 cardiomyocytes (20,21) illuminated with blue light, the inventors will transfect them with FP-LILAC. To check for successful transfection, the inventors will compare cells visible with DIC and cells expressing the FP. Because Lifeact has been shown to compete for binding with myosin through a shared actin binding site (22,23), by uncaging LILAC, the inventors hypothesize that this will reduce the amount of bound myosin, thus stalling beating. Fast Fourier transform (FFT) analysis of videos of beating cells has been previously used to quantify cardiomyocyte beating (66-68). If this strategy is successful, the beating amplitude and/or frequency should reduce in the location where LILAC is excited.

Next, the inventors would like to determine how mechanical force affects cardiomyocyte synchronization. Nitsan et al showed that cardiomyocytes can be trained to synchronize with mechanical probes mimicking neighboring cells (69). This effect lasts for an entire hour post stimulation, unlike electrical stimulation, suggesting long term effects of deformation. Additionally, actomyosin contractility was necessary for this process, but the spatiotemporal effect of actomyosin contractility was not explored (69-70). To address this, the inventors will illuminate a group of cells with the intent of stalling them in a ring of varying size and determine the size at which cells inside and outside the ring no longer beat synchronously through FFT video analysis. To investigate the effect of temporally modulating contractility, the inventors will pulse the rings of light and examine synchronicity.

### B. References

The following references, to the extent that they provide exemplary procedural or other details supplementary to those set forth herein, are specifically incorporated herein by reference.
1. Blanchoin L. Boujemaa-Paterski R. Sykes C. Plastino J. (2014). Actin Dynamics, Architecture, and Mechanics in Cell Motility. Physiological Reviews, 94 (1), 235-263
2. Stone, O. J., Pankow, N., Liu, B. et al. (2019). Optogenetic control of cofilin and αTAT in living cells using Z-lock. Nat Chem Biol 15, 1183-1190. https://doi.org/10.1038/s41589-019-0405-4.
3. Hughes, R. M., & Lawrence, D. S. (2014). Optogenetic engineering: light-directed cell motility. Angewandte Chemie (International ed. in English), 53 (41), 10904-10907. doi: 10.1002/anie.201404198
4. Kennis, J., Van Stokkum, I., Crosson, S., Gauden, M., Moffat, K., van Grondelle, R. (2004). The LOV2 Domain of Phototropin: A Reversible Photochromic Switch. Journal of the American Chemical Society. 126. 4512-3. 10.1021/ja031840r.
5. Crosson, S., & Moffat, K. (2002). Photoexcited structure of a plant photoreceptor domain reveals a light-driven molecular switch. The Plant cell, 14 (5), 1067-1075. doi: 10.1105/tpc.010475
6. Crosson, S., & Moffat, K. (2001). Structure of a flavin-binding plant photoreceptor domain: insights into light-mediated signal transduction. PNAS, 98 (6), 2995-3000. doi: 10.1073/pnas.051520298
7. Riedl, J., et al., (2008). Lifeact: a versatile marker to visualize F-actin. Nature methods, 5 (7), 605-607. doi: 10.1038/nmeth.1220
8. Zayner, J., Mathes, T., Sosnick, T., Kennis, J. (2019). Helical Contributions Mediate Light-Activated Conformational Change in the LOV2 Domain of Avena sativa Phototropin 1. ACS Omega. 4. 1238-1243. 10.1021/acsomega.8b02872.
9. Strickland, D., Lin, Y., Wagner, E., Hope, C. M., Zayner, J., Antoniou, C., . . . Glotzer, M. (2012). TULIPs: tunable, light-controlled interacting protein tags for cell biology. Nature methods, 9 (4), 379-384. doi: 10.1038/nmeth.1904
10. French, A. R., Sosnick, T. R., & Rock, R. S. (2017). Investigations of human myosin VI targeting using optogenetically controlled cargo loading. Proceedings of the National Academy of Sciences of the United States of America, 114 (9), E1607-E1616. doi: 10.1073/pnas.1614716114
11. Niopek, D., Benzinger, D., Roensch, J., Draebing, T., Wehler, P., Eils, R., & Di Ventura, B. (2014). Engineering light-inducible nuclear localization signals for precise spatiotemporal control of protein dynamics in living cells. Nature communications, 5, 4404. doi: 10.1038/ncomms5404
12. Lungu, O. I., Hallett, R. A., Choi, E. J., Aiken, M. J., Hahn, K. M., & Kuhlman, B. (2012). Designing photoswitchable peptides using the AsLOV2 domain. Chemistry & biology, 19 (4), 507-517. doi: 10.1016/j.chembiol.2012.02.006
13. Guntas, G., Hallett, R. A., Zimmerman, S. P., Williams, T., Yumerefendi, H., Bear, J. E., & Kuhlman, B. (2015). Engineering an improved light-induced dimer (iLID) for controlling the localization of signaling proteins. PNAS, 112 (1), 112-117. doi: 10.1073/pnas.1417910112
14. Strickland, Devin & Moffat, Keith & Sosnick, Tobin. (2008). Light-activated DNA binding in a designed allosteric protein. PNAS, 105. 10709-14. 10.1073/pnas.0709610105.
15. Tas, R. P., Chen, C. Y., Katrukha, E. A., Vleugel, M., Kok, M., Dogterom, M., . . . Kapitein, L. C. (2018). Guided by Light: Optical Control of Microtubule Gliding Assays. Nano letters, 18 (12), 7524-7528. doi: 10.1021/acs.nanolett.8b03011
16. Oakes, P. W., Wagner, E., Brand, C. A., Probst, D., Linke, M., Schwarz, U. S., . . . Gardel, M. L. (2017). Optogenetic control of RhoA reveals zyxin-mediated elasticity of stress fibres. Nature communications, 8, 15817. doi: 10.1038/ncomms15817
17. Zayner, J. P., & Sosnick, T. R. (2014). Factors that control the chemistry of the LOV domain photocycle. PloS one. 9 (1), e87074. doi: 10.1371/journal.pone.0087074.
18. Letort, G., Politi, A. Z., Ennomani, H., Théry, M., Nedelec, F., & Blanchoin, L. (2015). Geometrical and mechanical properties control actin filament organization. PLoS computational biology, 11 (5), e1004245. doi: 10.1371/journal.pcbi. 1004245
19. Gressin, Laurène & Guillotin, Audrey & Guerin, Christophe & Blanchoin, Laurent & Michelot, Alphée. (2015). Architecture Dependence of Actin Filament Network Disassembly. Current Biology. 25. 10.1016/j.cub.2015.04.011.
20. Claycomb, W., Lanson, N., Stallworth, B., Egeland, D., Delcarpio, J., Bahinski, A., & Izzo, N. (1998). HL-1 cells: A cardiac muscle cell line that contracts and retains phenotypic characteristics of the adult cardiomyocyte. Proceedings of the National Academy of Sciences, 95 (6), 2979-2984.
21. White, S., Constantin, P., & Claycomb, W. (2004). Cardiac physiology at the cellular level: use of cultured HL-1 cardiomyocytes for studies of cardiac muscle cell structure and function. American Journal of Physiology-Heart and Circulatory Physiology, 286 (3), H823-H829.
22. Belyy A, Merino F, Sitsel O, Raunser S (2020) Structure of the Lifeact-F-actin complex. PLoS Biol 18 (11): e3000925. https://doi.org/10.1371/journal.pbio.3000925
23. Kumari, A., Kesarwani, S., Javoor, M., Vinothkumar, K., & Sirajuddin, M. (2020). Structural insights into actin filament recognition by commonly used cellular actin markers. The EMBO Journal, 39 (14), e104006.
24 Boccafoschi, F., Mosca, C., Ramella, M., Valente, G., & Cannas, M. (2013). The effect of mechanical strain on soft (cardiovascular) and hard (bone) tissues: common pathways for different biological outcomes. Cell adhesion & migration, 7 (2), 165-173. https://doi.org/10.4161/cam.23020
25. Voorhees, A. P., & Han, H. C. (2015). Biomechanics of Cardiac Function. Comprehensive Physiology, 5 (4), 1623-1644. https://doi.org/10.1002/cphy.c140070
26. Oda, T., Namba, K., & Maéda, Y. (2005). Position and orientation of phalloidin in F-actin determined by X-ray fiber diffraction analysis. Biophysical journal, 88 (4), 2727-2736. doi: 10.1529/biophysj. 104.047753
27. Aizawa H., Sameshima M., Yahara I. (2006) A Green Fluorescent Protein-actin Fusion Protein Dominantly Inhibits Cytokinesis, Cell Spreading, and Locomotion in Dictyostelium, Cell Structure and Function, 22 (3), 335-345.
28. Melak, Michael & Plessner, Matthias & Grosse, Robert. (2017). Actin visualization at a glance. Journal of cell science. 130. 10.1242/jcs.189068.
29 Courtemanche, N., Pollard, T. D., & Chen, Q. (2016). Avoiding artefacts when counting polymerized actin in live cells with Lifeact fused to fluorescent proteins. Nature cell biology, 18 (6), 676-683. doi: 10.1038/ncb3351
30. Flores, L. R., Keeling, M. C., Zhang, X. et al. (2019). Lifeact-TagGFP2 alters F-actin organization, cellular morphology and biophysical behaviour. Sci Rep 9, 3241. https://doi.org/10.1038/s41598-019-40092-w
31. Christie, J. M. et al. (1999) LOV (light, oxygen, or voltage) domains of the blue-light photoreceptor phototropin (nph1): Binding sites for the chromophore flavin mononucleotide. PNAS. 96 (15): 8779-8783.
32. Sakai T. et al. (2001) Arabidopsis nph1 and npl1: blue light receptors that mediate both phototropism and chloroplast relocation. PNAS. 98:6969-6974.
33. Halavity H. S. & Moffat K. (2007). N- and C-Terminal Flanking Regions Modulate Light-Induced Signal Transduction in the LOV2 Domain of the Blue Light Sensor Phototropin 1 from Avena sativa, Biochemistry. 49, 14001-14009.
34. Zayner, J. P., Antoniou, C., French, A. R., Hause, R. J., Jr, & Sosnick, T. R. (2013). Investigating models of protein function and allostery with a widespread mutational analysis of a light-activated protein. journal, Biophysical 105 (4), 1027-1036. doi: 10.1016/j.bpj.2013.07.010
35. Yee, E. F. et al. (2015). Signal transduction in light-oxygen-voltage receptors lacking the adduct-forming cysteine residue. Nat. Commun. 6, 10079.
36. Harper, S. M., Christie, J. M. & Gardner, K. H. (2004) Disruption of the LOV-J R helix interaction activates phototropin kinase activity. Biochemistry 43, 16184-16192.
37. Harper, S. M., Neil, L. C. & Gardner, K. H. (2003) Structural basis of a phototropin light switch. Science 301, 1541-1544.
38. Yao, X., Rosen, M. K., & Gardner, K. H. (2008). Estimation of the available free energy in a LOV2-J alpha photoswitch. Nature Chemical Biology, 4 (8), 491-497. doi: 10.1038/nchembio.99
39. Srivastava, J., & Barber, D. (2008). Actin co-sedimentation assay; for the analysis of protein binding to F-actin. Journal of visualized experiments: JoVE, (13), 690. https://doi.org/10.3791/690
40. Heier, J. A., Dickinson, D. J., & Kwiatkowski, A. V. (2017). Measuring Protein Binding to F-actin by Co-sedimentation. Journal of visualized experiments: JoVE, (123), 55613. https://doi.org/10.3791/55613
41. Jansen, S., Collins, A., Yang, C., Rebowski, G., Svitkina, T., & Dominguez, R. (2011). Mechanism of actin filament bundling by fascin. The Journal of Biological Chemistry, 286 (34), 30087-30096. https://doi.org/10.1074/jbc.M111.251439
42. Winkelman, J. D., Suarez, C., Hocky G. M., Harker, A. J., Morganthaler, A. N., Christensen, J. R., Voth, G, A., Bartles, J. R., & Kovar, D. R. (2016). Fascin- and α-Actinin-Bundled Networks Contain Intrinsic Structural Features that Drive Protein Sorting. Current Biology, 26 (20), 2697-2706.
43. David S. Courson, & Ronald S. Rock (2010). Actin Cross-link Assembly and Disassembly Mechanics for α-Actinin and Fascin. Journal of Biological Chemistry, 285 (34), 26350-26357.
44. Foley, K. S., & Young, P. W. (2014). The non-muscle functions of actinins: an update. The Biochemical Journal, 459 (1), 1-13. https://doi.org/10.1042/BJ20131511
45. Ribeiro, E., et. al. (2014). The structure and regulation of human muscle α-actinin. Cell, 159 (6), 1447-1460. https://doi.org/10.1016/j.cell.2014.10.056
46. Bartles J. R. (2000). Parallel actin bundles and their multiple actin-bundling proteins. Current Opinion in Cell Biology, 12 (1), 72-78. https://doi.org/10.1016/s0955-0674 (99) 00059-9
47. Michelot, A., & Drubin, D. G. (2011). Building distinct actin filament networks in a common cytoplasm. Current Biology CB, 21 (14), R560-R569. https://doi.org/10.1016/j.cub.2011.06.019
48. Kadzik, R., Homa, K., & Kovar, D. (2020). F-Actin Cytoskeleton Network Self-Organization Through Competition and Cooperation. Annual Review of Cell and Developmental Biology, 36 (1), 35-60.
49. Gressin, L., Guillotin, A., Guerin, C., Blanchoin, L., & Michelot, A. (2015). Architecture dependence of actin filament network disassembly. Current Biology, 25 (11), 1437-1447. https://doi.org/10.1016/j.cub.2015.04.011
50. Letort, G., Politi, A. Z., Ennomani, H., Théry, M., Nedelec, F., & Blanchoin, L. (2015). Geometrical and mechanical properties control actin filament organization. PLoS Computational Biology, 11 (5), e1004245. https://doi.org/10.1371/journal.pcbi.1004245
51. Reymann, A. C., Martiel, J. L., Cambier, T., Blanchoin, L., Boujemaa-Paterski, R., & Théry, M. (2010). Nucleation geometry governs ordered actin networks structures. Nature Materials, 9 (10), 827-832. https://doi.org/10.1038/nmat2855
52. Reymann, A. C., et. al. (2012). Actin network architecture can determine myosin motor activity. Science, 336 (6086), 1310-1314. https://doi.org/10.1126/science. 1221708
53. Mansson et al. (2004) In vitro sliding of actin filaments labelled with single quantum dots. Biochem. and Biophys. Res. Comm., 314 (2), 529-534
54. Burkel, B. M., von Dassow, G., & Bement, W. M. (2007). Versatile fluorescent probes for actin filaments based on the actin-binding domain of utrophin. Cell Motility and the Cytoskeleton, 64 (11), 822-832. https://doi.org/10.1002/cm.20226
55. Brehm, M. A., Schreiber, I., Bertsch, U., Wegner, A. & Mayr, G. W. (2004). Identification of the actin-binding domain of Ins (1, 4, 5) P3 3-kinase isoform B (IP3K-B). Biochem J, 382, 353-362, https://doi.org/10.1042/BJ20031751
56. Zhiheng Jia, Virginijus Valiunas, Zongju Lu, Harold Bien, Huilin Liu, Hong-Zhang Wang, Barbara Rosati, Peter R. Brink, Ira S. Cohen, & Emilia Entcheva (2011). Stimulating Cardiac Muscle by Light. Circulation: Arrhythmia and Electrophysiology, 4 (5), 753-760.
57. Beiert, T., Bruegmann, T., & Sasse, P. (2014). Optogenetic activation of Gq signalling modulates pacemaker activity of cardiomyocytes. Cardiovascular Research, 102 (3), 507-516.
58. Entcheva, E., Kay, M. W. (2020). Cardiac optogenetics: a decade of enlightenment. Nat Rev Cardiol. https://doi-org.proxy.uchicago.edu/10.1038/s41569-020-00478-0
59. Wang, Y., Lin, W., Crawford, W. et al. (2017). Optogenetic Control of Heart Rhythm by Selective Stimulation of Cardiomyocytes Derived from Pnmt+ Cells in Murine Heart. Sci Rep 7, 40687 https://doi.org/10.1038/srep40687
60. Men, J., Li, A., Jerwick, J. et al. (2020). Non-invasive red-light optogenetic control of Drosophila cardiac function. Commun Biol 3, 336. https://doi.org/10.1038/s42003-020-1065-3
61. Saleh, M., & Ambrose, J. A. (2018). Understanding myocardial infarction. F1000Research, 7, F1000 Faculty Rev-1378. https://doi.org/10.12688/f1000research.15096.1
62. Doenst, T., Nguyen, T. D., & Abel, E. D. (2013). Cardiac metabolism in heart failure: implications beyond ATP production. Circulation Research, 113 (6), 709-724. https://doi.org/10.1161/CIRCRESAHA.113.300376
63. Robert C. Lyon, Fabian Zanella, Jeffrey H. Omens, & Farah Sheikh (2015). Mechanotransduction in Cardiac Hypertrophy and Failure. Circulation Research, 116 (8), 1462-1476.
64 Romito, E., Shazly, T., & Spinale, F. (2017). In vivo assessment of regional mechanics post-myocardial infarction: A focus on the road ahead. Journal of Applied Physiology, 123 (4), 728-745.
65. Pasqualini, F. S., Nesmith, A. P., Horton, R. E., Sheehy, S. P., & Parker, K. K. (2016). Mechanotransduction and Metabolism in Cardiomyocyte Microdomains. BioMed research international, 4081638. https://doi.org/10.1155/2016/4081638
66. Reno, A., Hunter, A. W., Li, Y., Ye, T., & Foley, A. C. (2018). Quantification of cardiomyocyte beating frequency using Fourier transform analysis. Photonics, 5 (4). https://doi.org/10.3390/photonics5040039
67. Hossain, M. M., Shimizu, E., Saito, M., Rao, S. R., Yamaguchi, Y., & Tamiya, E. (2010). Non-invasive characterization of mouse embryonic stem cell derived cardiomyocytes based on the intensity variation in digital beating video. The Analyst, 135 (7), 1624-1630. https://doi.org/10.1039/c0an00208a
68. Bazan, C., Torres Barba, D., Blomgren, P., & Paolini, P. (2011). Image processing techniques for assessing contractility in isolated neonatal cardiac myocytes. International Journal of Biomedical Imaging, 2011, 1-9. https://doi.org/10.1155/2011/729732
69. Nitsan, I., Drori, S., Lewis, Y. et al. (2016). Mechanical communication in cardiac cell synchronized beating. Nature Phys 12, 472-477. https://doi.org/10.1038/nphys3619
70. Cohen, O., & Safran, S. A. (2018). Theory of frequency response of mechanically driven cardiomyocytes. Scientific reports, 8 (1), 2237. https://doi.org/10.1038/s41598-018-20307-2
71. Zhang, J., Wilson, G. F., Soerens, A. G., Koonce, C. H., Yu, J., Palecek, S. P., Thomson, J. A., & Kamp, T. J. (2009). Functional cardiomyocytes derived from human induced pluripotent stem cells. Circulation Research, 104 (4), e30-e41. https://doi.org/10.1161/CIRCRESAHA.108.192237
72. Pushp, P., Sahoo, B., Ferreira, F., Sampaio Cabral, J., Fernandes-Platzgummer, A., & Gupta, M. (2020). Functional comparison of beating cardiomyocytes differentiated from umbilical cord-derived mesenchymal/stromal stem cells and human foreskin-derived induced pluripotent stem cells. Journal of Biomedical Materials Research, 108 (3), 496-514.

### Example 2: LILAC: Enhanced Actin Imaging with an Optogenetic Lifeact

### A. Introduction and Results

The inventors have designed an improved Lifeact variant that binds to actin under the control of light using the LOV2 protein. Light control enables one to subtract the pre-illumination signal of the unbound label, yielding an enhanced view of F-actin dynamics in cells. Furthermore, the tool eliminates actin network perturbations and cell sickness caused by Lifeact overexpression.

The actin cytoskeleton is central to a range of cellular processes, from cellular adhesion, shape and movement, to endo- and exocytosis, and cytokinesis. Dependable actin visualization is vital for various fields of biological research¹. Phalloidin is the gold standard for actin filament visualization; however, its use is limited to fixed-staining due to its membrane impermeability. A number of tools exist for live-cell imaging of actin including fluorescent protein (FP) tagged actin monomers, Utrophin, F-Tractin, actin chromobodies and Lifeact². Of these imaging reagents, Lifeact, the first 17 amino acids of yeast actin binding protein 140 (ABP140), has become the most common label actin filaments in live cells, being cited in over 1800 papers³.

However, recent work has shown that Lifeact has unintended effects, such as Drosophila sterility, mesenchymal stem cell morphology changes, and altered actin patch and cytokinetic ring dynamics in fission yeast⁴⁻⁶. These side effects are likely due to competitive binding between Lifeact and other important proteins, such as cofilin and myosin, which also bind actin filaments at the D-loop^{7,8}. To address this serious problem and develop a new optogenetic tool, the inventors designed LILAC (light-induced localization to actin) that has low actin affinity in the dark. Upon excitation, the photo-activated Lifeact diffuses within a second from the cytosol to actin filaments, labeling them for imaging. LILAC photocycles and returns to the low affinity state after a minute which minimizes side effects. In addition, the light-controlled aspect greatly improves the imaging capability.

In this design, the inventors employed LOV2, the second light-sensitive light-oxygen-voltage (LOV) domain from phototropinI in oats which is involved in the phototropism response in plants. In response to blue light, the flavin chromophore forms an adduct with C450, resulting in conformational changes within the core^{9,10}. This change disrupts the seven residue N-terminal A′α helix and 24 residue C-terminal Jα helix, causing them to unfold¹¹. This light-dependent conformational change of LOV2 has been harnessed to engineer a variety of optogenetics tools¹²⁻¹⁶.

The dark state recovery of LOV2 has a time constant of 81 sec for the wild type protein at room temperature and pH 8¹⁷. This time constant is tunable over several orders of magnitude through mutations and buffer conditions¹⁸⁻²¹. Additionally, mutations to mimic constitutive light-exposed, unfolded (1539E) and the dark, folded state (C450A) are well characterized²²⁻²⁴.

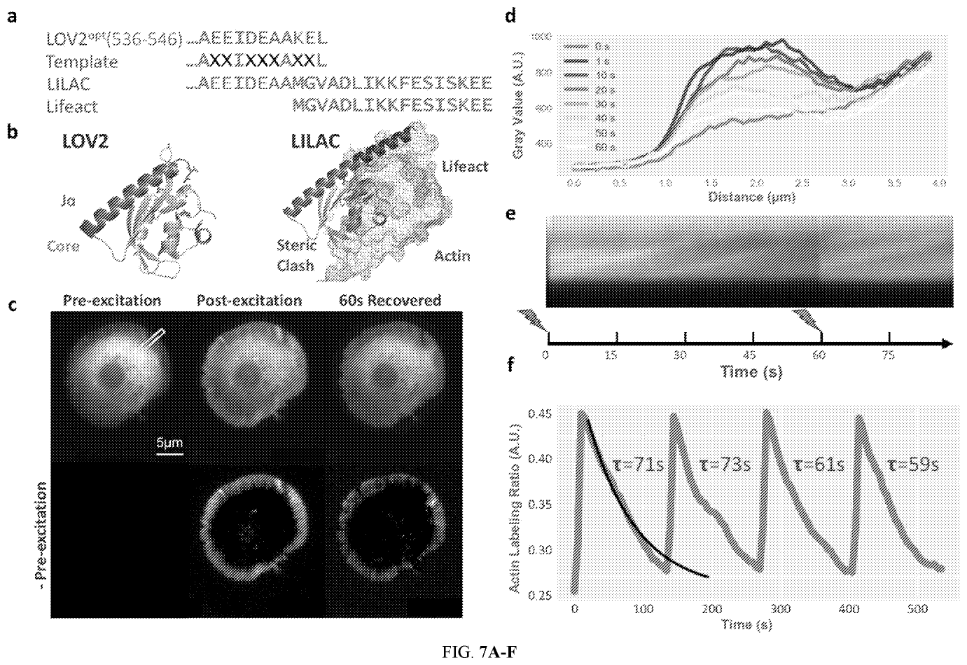

Using previous LOV2-based optogenetic platforms as a guide and minimizing disruption of the hydrophobic contacts between the Jα helix and the core of LOV2, the inventors designed LILAC (FIG. 7a). This design is intended to sterically prevent binding to actin by caging Lifeact, when the Jα helix is folded (FIG. 7b). This caging is achieved by positioning the hydrophobic residues of Lifeact, which are necessary for actin binding in cells^{7,8}, towards the core of LOV2. Upon unfolding of the Jα helix, Lifeact is uncaged and able to bind actin filaments.

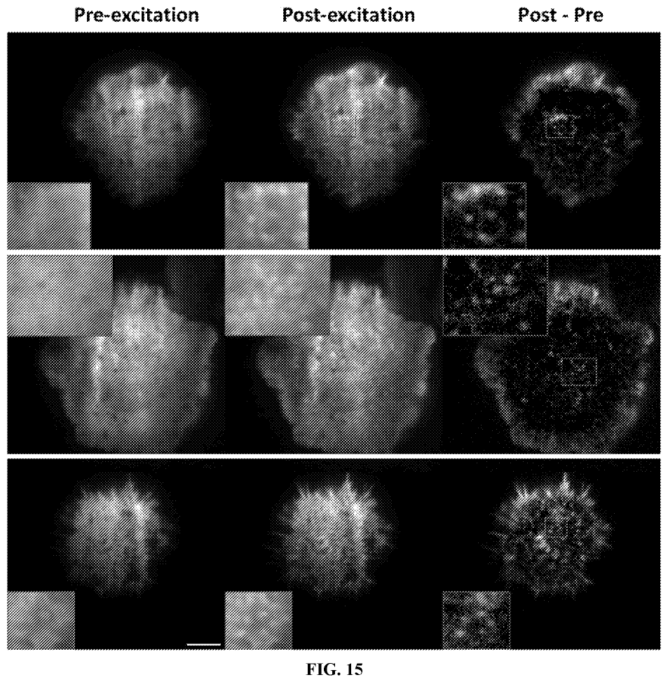

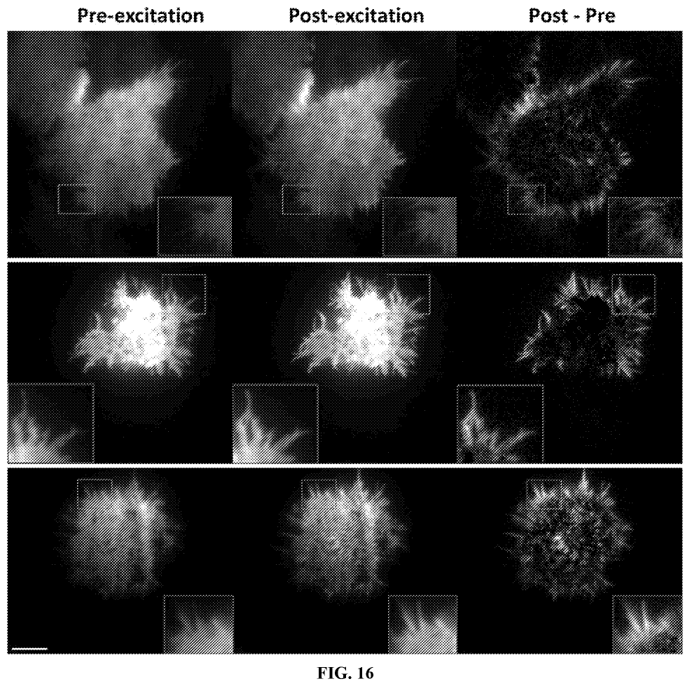

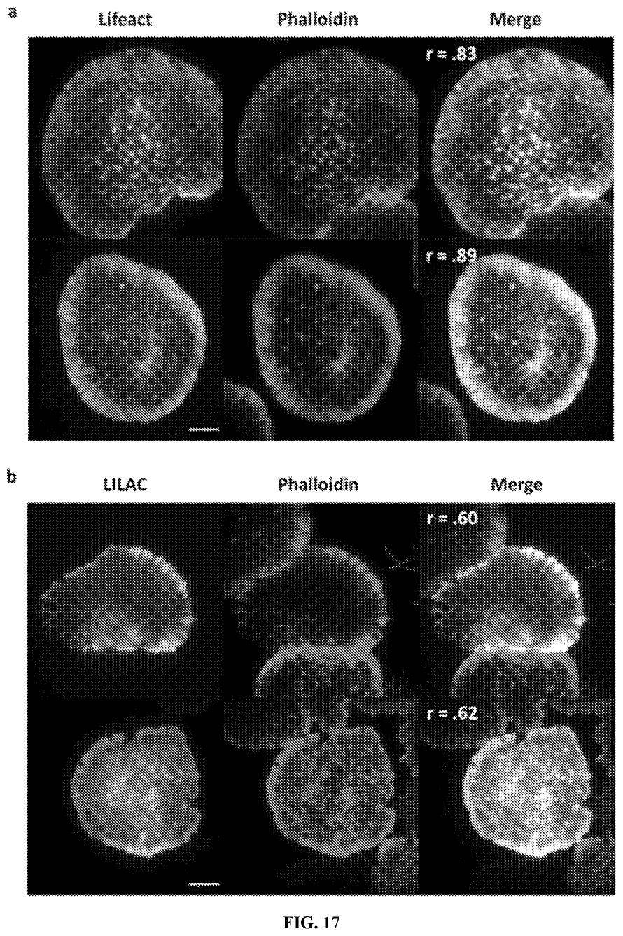

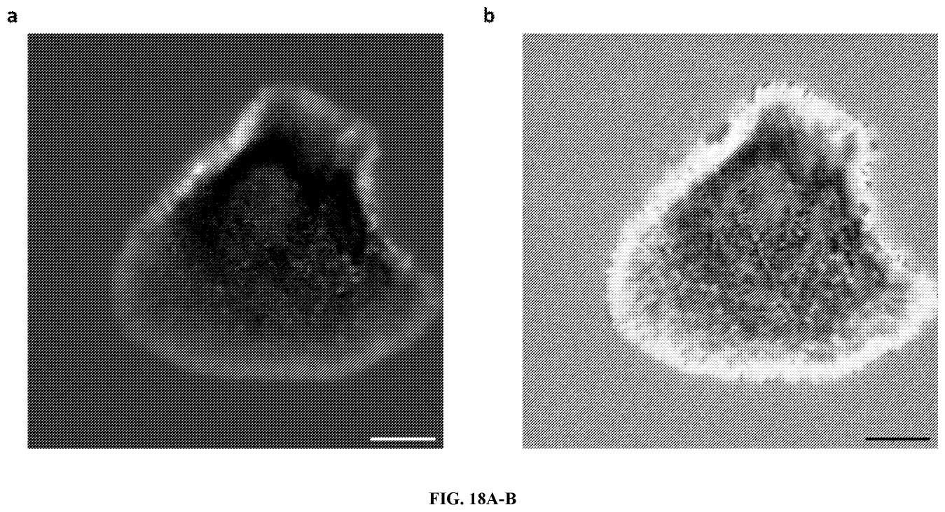

The inventors used TIRF imaging to test filament labeling in the light and dark. After blue light excitation, D. melanogaster S2 cells have increased fluorescence in the outer lamellipodial actin ring as compared to the dark state (FIG. 7c). This increased lamellipodial fluorescence reverses with a time constant similar to WT LOV2, suggesting that the reversion of the LOV2 domain to its dark state drives unbinding from the actin (FIG. 7d). Further, by subtracting the pre-excitation image, the inventors effectively eliminate the background fluorescence from the cytosolic, unbound LILAC. This subtraction yields a substantially improved image that highlights the structures of interest (FIG. 7c, FIG. 9a-b), increases the image contrast in the lamellipodium 2.5 fold (FIG. 9e), and reduces the influence of variations in cellular thickness. Subtraction of the pre-excitation image also enhances the appearance of retrograde flow in kymographs (FIG. 7e, FIG. 9c-d). These enhanced kymographs show retrograde flow of actin at a rate of 3.5±0.12 μm/min, which is similar to the rate of 4.0±0.44 found by Rogers²⁵. Beyond lamellipodia, post-excitation images also reveal small patches in the cell body (FIG. 15), as well as filopodia in landing S2 cells (FIG. 16). Staining S2 cells expressing either LifeAct or LILAC with phalloidin confirmed that central and peripheral structures labeled by LILAC are actin-rich (FIG. 17). Moreover, LILAC coupled with pulsed excitation enables other enhanced imaging modes such as OLID²⁶ (FIG. 18), which reveals regions of correlated actin labeling. A single excitation pulse train can be analyzed by either background subtraction or OLID, depending upon user need.

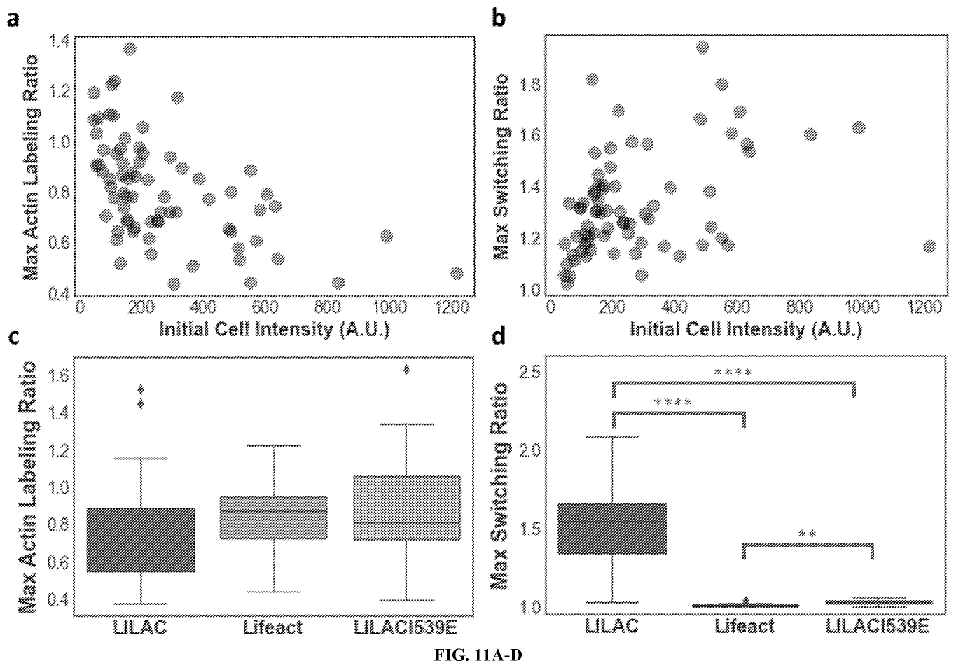

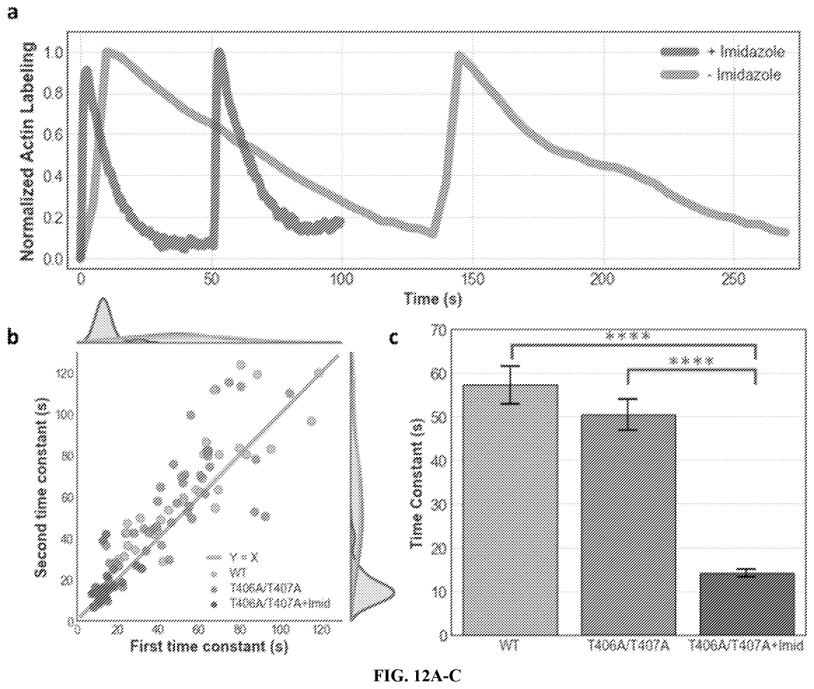

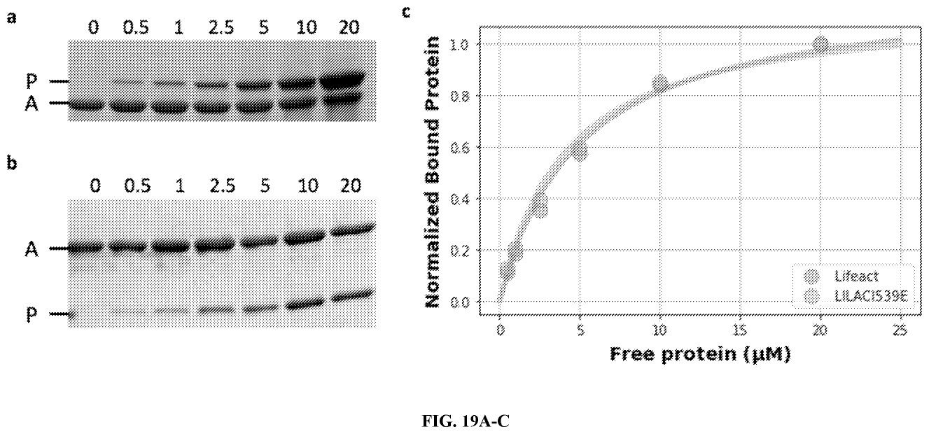

To quantify the images, the inventors used the actin labeling ratio (ALR), defined as the ratio of average fluorescence in the outer ring, divided by the average fluorescence of the inside of the cells over time (FIG. 7f, 10). S2 cells expressing larger amounts of LILAC tend to show lower maximum ALRs as cell intensity decreases (R2=0.34, p=2.2e-12, FIG. 11a), likely due to increased background from excess cytosolic LILAC. They found that the maximum ALRs of LILAC are similar to Lifeact, implying that the activated LILAC binds F-actin with a similar affinity and specificity as Lifeact (FIG. 11c). To confirm this, the inventors expressed and purified mCherry-Lifeact and mCherry-LILACI593E and determined their binding constants with F-actin through cosedimentation. This 1593E mutation forces the unfolding of the Jα helix, rendering the LILACI539E constitutively active^{13,22-24}. They found that Lifeact and LILACI539E bind with dissociation constants (K_{d}) of 3.9 μM and 3.4 μM, respectively (FIG. 19), between the previous reported Lifeact dissociation constants of 2.0 μM³ and 14.9 μM⁷. They further define the switching ratio as the fold change of the actin labeling ratio (post/pre-excitation). LILAC has a dark vs. light switching ratio of ˜1.6, while expectedly, Lifeact and LILACI539E exhibit no change in the light with switching ratios of ˜1 (FIG. 11d). The inventors find that the maximum switching ratio does not correlate strongly with cell intensity (R²=0.13, p=1.8e-5, FIG. 11b). Therefore, the on/off switching capabilities of LILAC enable an imaging in cells overexpressing LILAC, unlike Lifeact.

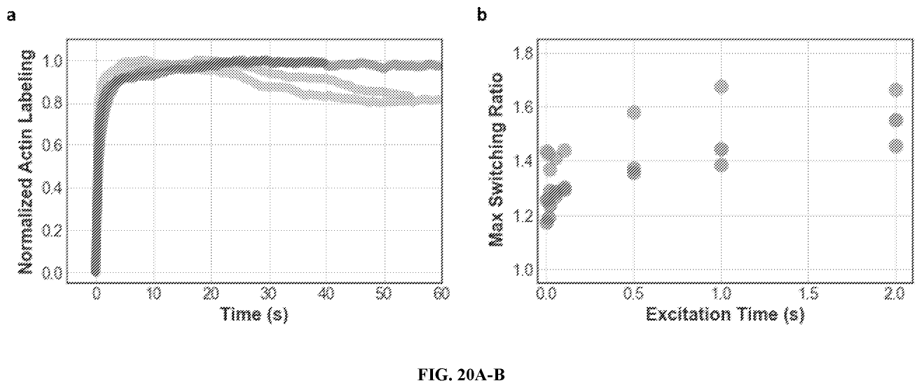

The LILAC labeling of the lamellipodium decays over time, implying reversible, light-activated labeling. The ALR sharply increases immediately after excitation, followed by the recovery back to nearly pre-excitation levels. This photo-excitation can be repeated multiple times, with no reduction in efficacy (FIG. 7f). To test sustained actin-labeling, the inventors continually excited cells for up to 60 seconds, with little reduction in actin labeling (FIG. 20a). Additionally, the inventors varied excitation time to determine its effect on the switching ratio. They found that the maximum switching ratio increases with increased excitation time, but saturates between 500 ms to 1 s (FIG. 20b).

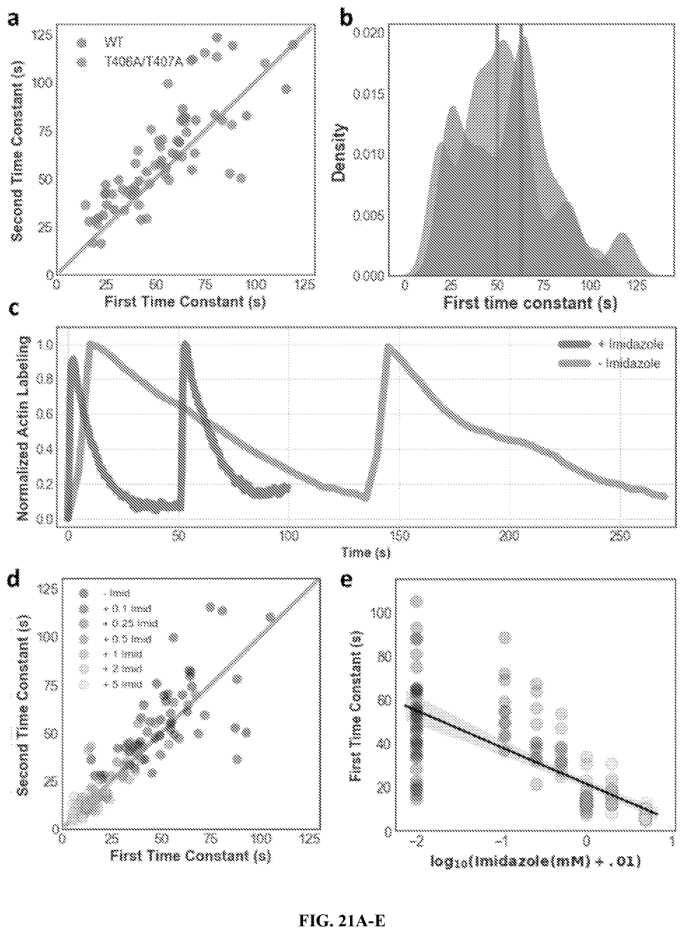

The inventors next examined the dark state recovery process to determine how rapidly the cell will reset after imaging. From actin labeling ratio traces, they determine the recovery time constant associated with each cell. On average, cells recover with a time constant of 63 seconds, close to the characteristic 80 second in vitro time constant of LOV211 (FIG. 21a,b). The inventors repeated this measurement with the T406A/T407A mutation, which stabilizes the N-terminal A′α helix, reduces the recovery time constant, and can increase caging^{12,17}. The average time constant decreased to 50 seconds, similar to the 54 second time constant measured previously in vitro¹⁷ (FIG. 21a,b).

Unexpectedly, the inventors observed a considerable amount of cell-to-cell variance in the recovery time, but for a given cell, the first and second recovery times were strongly correlated (R²=0.64, p=2.4e-17, FIG. 21a). This correlation suggests that each cell has an intrinsic time constant that is linked to cellular state (e.g., metabolite concentrations or pH), given that LOV2 dark state recovery is sensitive to buffer conditions^{20,21}. To determine whether the inventors could easily perturb these recovery times, they added imidazole to cell media, as LOV2 recovery time has previously been shown to be reduced by imidazole in vitro²¹. Similar to these in vitro results, they found that the in vivo recovery time constant is greatly dependent on imidazole (R²=0.52, p=3.5e-24, FIG. 21c-e). Addition of imidazole decreases the dark state recovery times and cell-to-cell variance, without affecting the intracellular correlation between time constants (R²=0.77, p=7.3e-46, FIG. 21d). The strong dependence of LILAC reversal on imidazole suggests that unbinding of LILAC from LOV2 is concurrent with the refolding of the LOV2 domain. It also shows that imidazole can effectively be used to enhance dark-state recovery, which can be useful in generating background-subtracted or OLID movies with multiple excitation cycles.

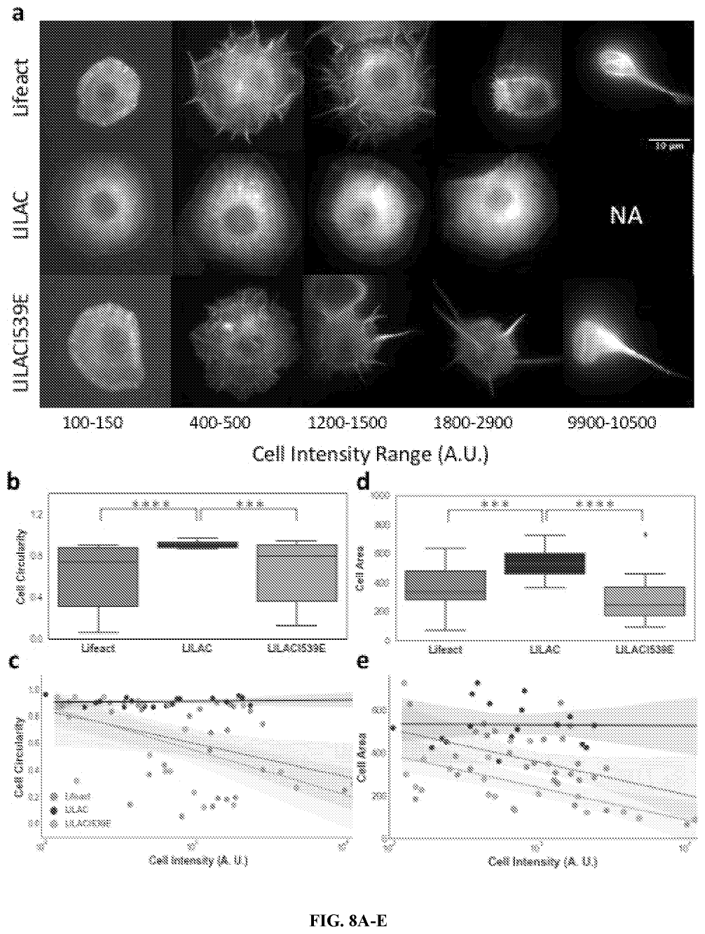

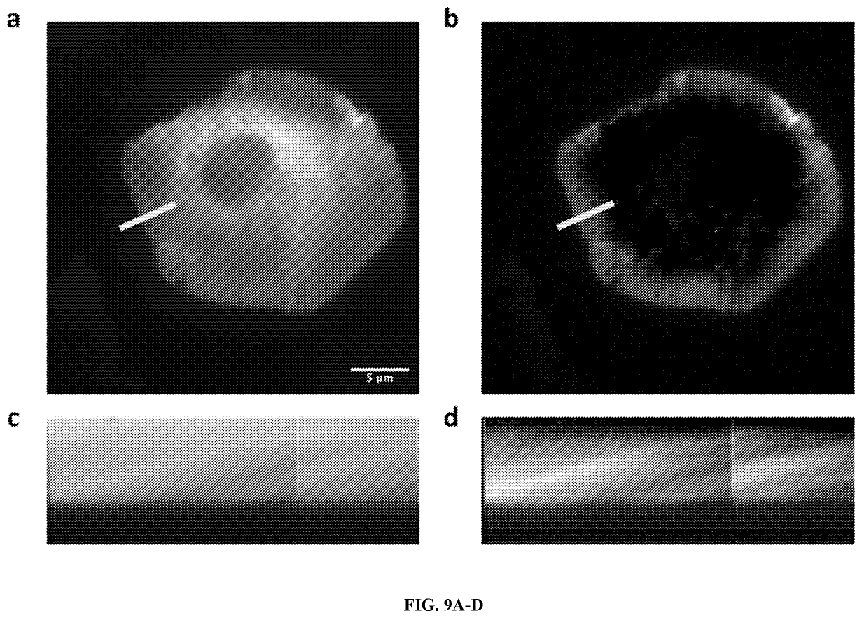

Next, the inventors examined the effects of high mCherry-Lifeact expression levels on the morphology of insect S2 cells using total internal reflection fluorescence microscopy (TIRF). They found that at low expression levels, cells are round with the characteristic lamellipodial actin ring at the outer edge. However, at increasing concentrations of mCherry-Lifeact, however, cells begin to form long F-actin bundles and protrusions (FIG. 8a). Such cells have an irregular shape and have impaired spreading onto the coverslips. This enhanced F-actin assembly could be due to the competition between Lifeact and cofilin⁷, which in turn reduces F-actin turnover.

LILAC greatly reduces these morphological effects induced by Lifeact expression. At the highest level of expression, LILAC expressing cells maintain their round shape and lack the prominent F-actin bundles (FIG. 8a). In addition to the circularity being significantly reduced with Lifeact in comparison to LILAC (P=1.57*10⁻⁴, FIGS. 8b,c), the inventors also observe that circularity depends more on LifeAct expression level (R²=0.12) than it does on LILAC expression level (R²=0.012). Lifeact expressing cells also have reduced spread area as a function of expression level (R²=0.21). This effect is absent when expressing LILAC (R²=2.8*10⁻⁴, FIGS. 8d,e). These observations quantitatively suggest that Lifeact alters the actin cytoskeleton and the resulting cell morphology whereas LILAC does not.

To test whether the lack of cytoskeletal artifacts in LILAC-labeled cells was due to its ability to effectively cage the Lifeact peptide, the inventors evaluated circularity and cell area in a LILAC containing the 1539E mutation in the LOV2 domain. As expected, LILACI539E recapitulates the morphological defects seen in S2 cells overexpressing mCherry-Lifeact (FIG. 8a). Similarly to mCherry-Lifeact, circularity (R²=0.40, FIGS. 8b,c) and cell area (R²=0.36, FIGS. 8d,e) correlate with expression level. Overall, these results show that LILAC is nearly inert in the dark, even when highly overexpressed, and that this is likely due to effective caging of the Lifeact peptide.

In summary, LILAC has great potential utility as a new tool for actin imaging. By subtracting the pre-excitation image, the inventors eliminate the cytosolic background and obtain superior images of the actin cytoskeleton alone. For cytosolic background subtraction, current methods require a separate cytosolic fluorescent label, but LILAC allows for background subtraction with a single construct. LILAC can be tagged with various fluorophores depending on user needs. For example, if tagged with a green fluorescent protein (GFP), excitation of the fluorophore and LOV2 would be simultaneous. This allows for the use of a single excitation line, but eliminates the option of pre-excitation image subtraction. Alternatively, GFP may be used to label a second protein of interest, which can be imaged with the understanding that such imaging will also photoconvert the LILAC. An image sequence of 1) pre-excitation LILAC, 2) GFP-POI, 3) post-excitation LILAC would allow for the same background subtraction as in FIG. 7c. Thus, LILAC can be used in multiple ways depending on the need, and the end user is in the best position to evaluate the tradeoffs. Recovery times can be tuned by adjusting solvent conditions, allowing for slow recovery for recording movies of actin dynamics, or rapid recovery when minimal perturbation is required. Moreover, LILAC eliminates Lifeact induced side-effects when cells are grown in the dark. In the future, the inventors envision that a spatiotemporally controlled actin binding protein can be used to control actin cross-linking and patterning, and the subcellular recruitment of actin binding proteins.

### B. Methods

### 1. DNA Preparation

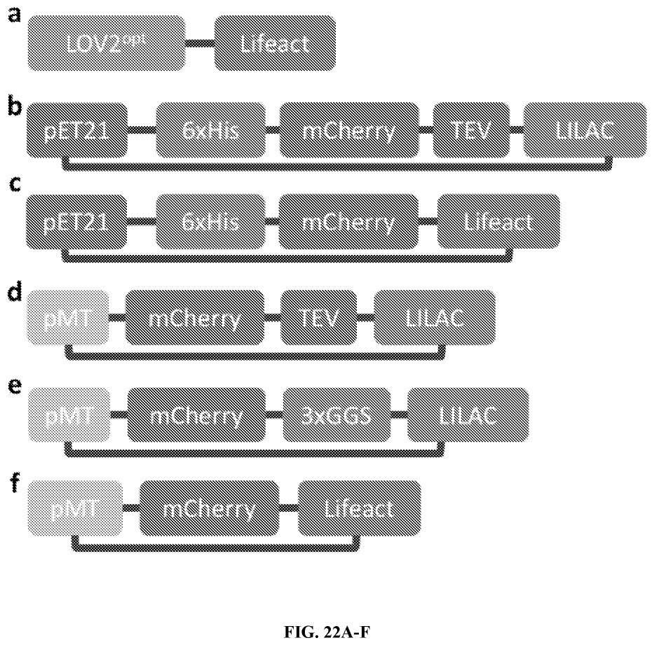

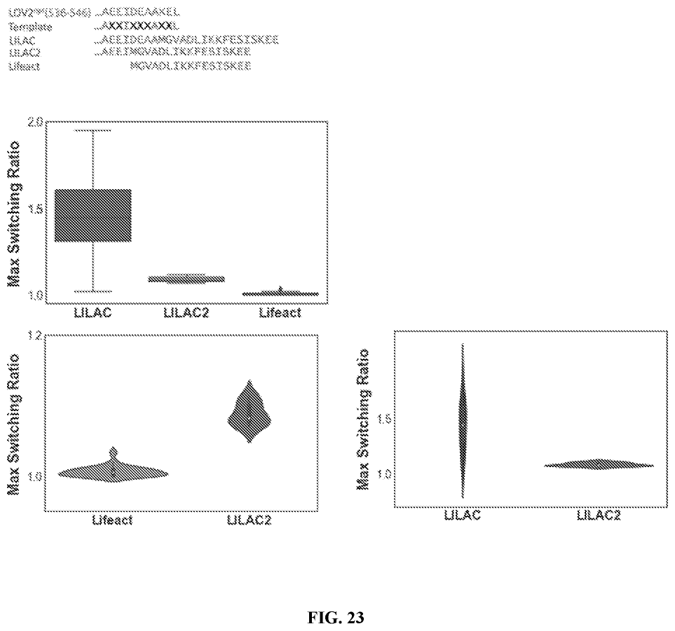

A LILAC G-block was purchased from Integrated DNA Technologies and incorporated into a pet21 vector containing 6×His-mCherry-TEV using Gibson Assembly containing AsLOV2 (401-543) with the mutations G528A/1532A/N538E for improved switching¹ to make LOV2^{opt} (FIG. 22a,b). LOV2 was removed to create the pET21-mCherry-Lifeact vector using the NEB Q5 protocol (FIG. 22c). Next, the inventors used Gibson assembly to incorporate mCherry-TEV-LILAC into a pMT vector from the Glotzer laboratory (UChicago) (FIG. 22d). The TEV protease site was then replaced with a 3×GGS linker using the NEB Q5 protocol (FIG. 22e). LOV2 was removed to create the pMT-mCherry-Lifeact vector using the NEB Q5 protocol (FIG. 22f). The T406A/T407A and I539E mutations were created using the NEB Q5 protocol.

### 2. Cell Maintenance

Insect S2 cells from the Fehon Lab (UChicago) were maintained in Schneider's insect media (Sigma) with insect media supplement (Sigma), 50 units/mL penicillin, and 50 μg/ml streptomycin (Thermo). Cells were split 1:6 every 4 to 6 days at around 10-15 million cells/ml at 20 ml in T75 flasks.

### 3. Transfection and Imaging Preparation

Cells were transfected as previously described². In short, 2 ml of fast-growing cells at 1.35 million/ml to a 6 well plate. DDAB at 250 μg/ml was added to the media in a 1:2 ratio and left for ten minutes. Then, the DDAB-media mixture was added to 500 ng of pMT vector to reach 150 μl and left for fifteen minutes. This was added dropwise to the cell. One day later, cells were induced with 1.5 μl of 0.7 M CuSO₄. Cells were transferred one day later on glass-bottom dishes coated with 15 μl of 0.5 mg/ml concanavalin A³. For FIG. 20, 1M pH 7.5 sterile filtered imidazole was added to media to reach the final desired concentration. Unless otherwise noted, after 45 minutes to allow for cell spreading, cells were imaged.

### 4. Fixation and Staining

Cells were transfected with either LILAC or Lifeact and spread onto ConA coated coverslips 1 day post induction. After removal of media, cells were rinsed with PBS and 4% paraformaldehyde (diluted in PBS) was added for 15 minutes under blue light. Cells were permeabilized with 0.2% Triton X-100 in PBS. After rinsing with PBS, cells were stained with Alexa Fluor 488 phalloidin and rinsed again with PBS.

### 5. Imaging

Dishes were imaged using a 100×, 1.65 N.A. objective (Olympus) on a custom-built total internal reflection microscope using an electron-multiplying charge-coupled device (EMCCD) camera (iXon; Andor Technologies) with a mCherry specific emission filter. This microscope was controlled with the open source Micro-Manager program. Unless otherwise noted, cells were excited first with 561 nm, then with 488 nm and 561 nm, and recovery images were taken with 561 nm excitation only, all at 1% power for 500 ms exposure with an EM gain of 200 with no delay time between images. This results in an irradiance of 1 Watt/cm² of blue light, which is 10-20× the level typically used to saturate LOV2⁴. Cells excited more than twice were imaged in exactly the same manner with 5 s between images instead of 500 ms. Cells in FIG. 8 were imaged on the same microscope on the same day to best guarantee cells of the same brightness are expressing similar protein levels.

### 6. Image Analysis

Outer and inner rings of the cells were traced by hand and kymographs were created in Fiji (available online at imagej.net). Inset images were created using a Fiji macro (available online at imagej.nih.gov/ij/macros/tools/Zoom_in_Images_and_Stacks.txt). Brightness, cell perimeter, area, circularity, and roundness values were exported from Fiji for inner and outer cell areas for each cell. From this, actin labeling ratio and switching ratios were calculated.

$$Actin Labeling Ratio ( A L R ) = \frac{{Intensity}_{outer}}{{Intensity}_{inner}}$$

$$Switching Ratio = \frac{A L {R}_{post}}{A L {R}_{pre}}$$

Time constants were calculated by fitting actin labeling ratio to a single exponential. Data analysis and figures were made using Python scripts.

The inventors quantified morphological changes by calculating the cell circularity, area, and roundness. When cells are circular, this value is 1, and when cells have large perimeters, it approaches 0. R² values were calculated using the logarithm of the cell intensity and the circularity/cell area.

$$Circularity = \frac{4 π * Area}{{Perimiter}^{2}} = \frac{4 π * ( π {r}^{2} )}{{( 2 * π r )}^{2}}$$

### 7. OLID Image Analysis

OLID analysis was performed in a similar manner to the original report⁵. We identified a rectangular region of the lamellipodium with LILAC enrichment after a blue light pulse, and summed this region to determine a LILAC response function. This response function had a similar rise and decay as shown in FIG. 7f. The inventors determined the Pearson correlation coefficient for each pixel (along time) with the response function, to generate the single static image shown in FIG. 18. To generate the related movie, we performed similar correlations using a sliding window over time (30 frames, offset 5 frames at a time), where the window was applied to both the response function and the source movie.

### 8. Protein Expression and Purification

Proteins were expressed and purified similar to previously described¹. In short, all proteins were expressed in Escherichia coli BL21 (DE3) cells grown in LB medium at 37° C. to an OD 600 nm of 0.6 and induced with 1 mM IPTG. After ˜18 hours in the dark, cells were pelleted and resuspended in 50 mM Tris (pH 8.0), 100 mM NaCl, 5 mM imidazole, 5% glycerol. Cells were lysed by sonication and clarified by centrifugation at 14,000 g for 40 minutes. Proteins were purified using metal affinity chromatography. After purification, the protein was dialyzed into assay buffer (25 mM KCl, 25 mM imidazole pH 7.5, 1 mM K-EGTA, 4 mM MgCl₂, 1 mM DTT).

### 9. Cosedimentation

Cosedimentation assays were performed as previously described⁶. Briefly, G-actin was polymerized, and filaments were stabilized with phalloidin. After preparation of the protein of interest by a hard spin to remove aggregates (100,000 g for 10 minutes), protein was incubated with 0.5 μM actin at room temperature for 60 minutes in assay buffer. After centrifugation at 100,000 g for 30 minutes, the supernatant was removed, and the pellet was resuspended before analyzing samples by SDS-PAGE. Gels were analyzed in Fiji (found online at imagej.net). To account for loading differences, pelleted protein was normalized by pelleted actin in each lane.

### C. References

The following references, to the extent that they provide exemplary procedural or other details supplementary to those set forth herein, are specifically incorporated herein by reference.
1 Blanchoin, L., Boujemaa-Paterski, R., Sykes, C. & Plastino, J. Actin dynamics, architecture, and mechanics in cell motility. Physiol. Rev. 94, 235-263 (2014).
2. Melak, M., Plessner, M. & Grosse, R. Actin visualization at a glance. Journal of Cell Science (2017) doi: 10.1242/jcs.189068.
3. Riedl, J. et al. Lifeact: a versatile marker to visualize F-actin. Nat. Methods 5, 605-607 (2008).
4. Spracklen, A. J., Fagan, T. N., Lovander, K. E. & Tootle, T. L. The pros and cons of common actin labeling tools for visualizing actin dynamics during Drosophila oogenesis. Dev. Biol. 393, 209-226 (2014).
5. Flores, L. R., Keeling, M. C., Zhang, X., Sliogeryte, K. & Gavara, N. Author Correction: Lifeact-TagGFP2 alters F-actin organization, cellular morphology and biophysical behaviour. Sci. Rep. 9, 9507 (2019).
6. Courtemanche, N., Pollard, T. D. & Chen, Q. Avoiding artefacts when counting polymerized actin in live cells with LifeAct fused to fluorescent proteins. Nat. Cell Biol. 18, 676-683 (2016).
7. Belyy, A., Merino, F., Sitsel, O. & Raunser, S. Structure of the Lifeact-F-actin complex. PLoS Biol. 18, e3000925 (2020).
8. Kumari, A., Kesarwani, S., Javoor, M. G., Vinothkumar, K. R. & Sirajuddin, M. Structural insights into actin filament recognition by commonly used cellular actin markers. EMBO J. 39, e104006 (2020).
9. Crosson, S. & Moffat, K. Structure of a flavin-binding plant photoreceptor domain: insights into light-mediated signal transduction. Proc. Natl. Acad. Sci. U.S.A 98, 2995-3000 (2001).
10. Kennis, J. T. M. et al. The LOV2 domain of phototropin: a reversible photochromic switch. J. Am. Chem. Soc. 126, 4512-4513 (2004).
11. Halavaty, A. S. & Moffat, K. N- and C-Terminal Flanking Regions Modulate Light-Induced Signal Transduction in the LOV2 Domain of the Blue Light Sensor Phototropin 1 from Avena sativa. Biochemistry vol. 46 14001-14009 (2007).
12. Strickland, D. et al. TULIPs: tunable, light-controlled interacting protein tags for cell biology. Nature Methods vol. 9 379-384 (2012).
13. French, A. R., Sosnick, T. R. & Rock, R. S. Investigations of human myosin VI targeting using optogenetically controlled cargo loading. Proceedings of the National Academy of Sciences vol. 114 E1607-E1616 (2017).
14. Niopek, D. et al. Engineering light-inducible nuclear localization signals for precise spatiotemporal control of protein dynamics in living cells. Nature Communications vol. 5 (2014).
15. Engineering an Improved Light-induced Dimer (iLID) for Controlling the Localization and Activity of Signaling Proteins. (United States. Department of Energy. Office of Basic Energy Sciences, 2014).
16. Strickland, D., Moffat, K. & Sosnick, T. R. Light-activated DNA binding in a designed allosteric protein. Proceedings of the National Academy of Sciences vol. 105 10709-10714 (2008).
17. Zayner, J. P., Antoniou, C. & Sosnick, T. R. The amino-terminal helix modulates light-activated conformational changes in AsLOV2. J. Mol. Biol. 419, 61-74 (2012).
18. Zayner, J. P., Antoniou, C., French, A. R., Hause, R. J. & Sosnick, T. R. Investigating Models of Protein Function and Allostery With a Widespread Mutational Analysis of a Light-Activated Protein. Biophysical Journal vol. 105 1027-1036 (2013).
19. Zayner, J. P., Mathes, T., Sosnick, T. R. & Kennis, J. T. M. Helical Contributions Mediate Light-Activated Conformational Change in the LOV2 Domain of Avena sativa Phototropin 1. ACS Omega vol. 4 1238-1243 (2019).
20. Zayner, J. P. & Sosnick, T. R. Factors that control the chemistry of the LOV domain photocycle. PLoS One 9, e87074 (2014).
21 Alexandre, M. T. A., Arents, J. C., van Grondelle, R., Hellingwerf, K. J. & Kennis, J. T. M. A Base-Catalyzed Mechanism for Dark State Recovery in the Avena sativa Phototropin-1 LOV2 Domain. Biochemistry vol. 46 3129-3137 (2007).
22. Harper, S. M., Neil, L. C. & Gardner, K. H. Structural Basis of a Phototropin Light Switch. Science vol. 301 1541-1544 (2003).
23. Matsuoka, D. & Tokutomi, S. Blue light-regulated molecular switch of Ser/Thr kinase in phototropin. Proceedings of the National Academy of Sciences vol. 102 13337-13342 (2005).
24. Harper, S. M., Christie, J. M. & Gardner, K. H. Disruption of the LOV-Jα Helix Interaction Activates Phototropin Kinase Activity. Biochemistry vol. 43 16184-16192 (2004).
25. Rogers, S. L., Wiedemann, U., Stuurman, N. & Vale, R. D. Molecular requirements for actin-based lamella formation in Drosophila S2 cells. J. Cell Biol. 162, 1079-1088 (2003).
26. Marriott, G. et al. Optical lock-in detection imaging microscopy for contrast-enhanced imaging in living cells. Proc. Natl. Acad. Sci. U.S.A 105, 17789-17794 (2008).

### Methods References

1. Strickland, D. et al. Rationally improving LOV domain-based photoswitches. Nat Methods 7, 623-626 (2010).
2. Kyuhyung Han. An efficient DDAB-mediated transfection of Drosophila S2 cells. Nucleic Acids Res. 24, 4362-4363 (1996).
3. Buster, D. et al. Preparation of Drosophila S2 cells for Light Microscopy. J. Vis. Exp. 40, e1982 (2010).
4. Ruijgrok, P. V. et al. Optical control of fast and processive engineered myosins in vitro and in living cells. Nat, Chem. Bio., 17 (5), (2021).
5. Marriott, G. et al. Optical lock-in detection imaging microscopy for contrast-enhanced imaging in living cells. Proc. Natl. Acad. Sci. U.S.A 105, 17789-17794 (2008).
6. Heier, J. A., Dickinson, D. J., & Kwiatkowski, A. V. Measuring Protein Binding to F-actin by Co-sedimentation. JoVE, 123, 55613. (2017).

### Example 3: Expression of Optogenetic Polypeptides in Mammalian Cells

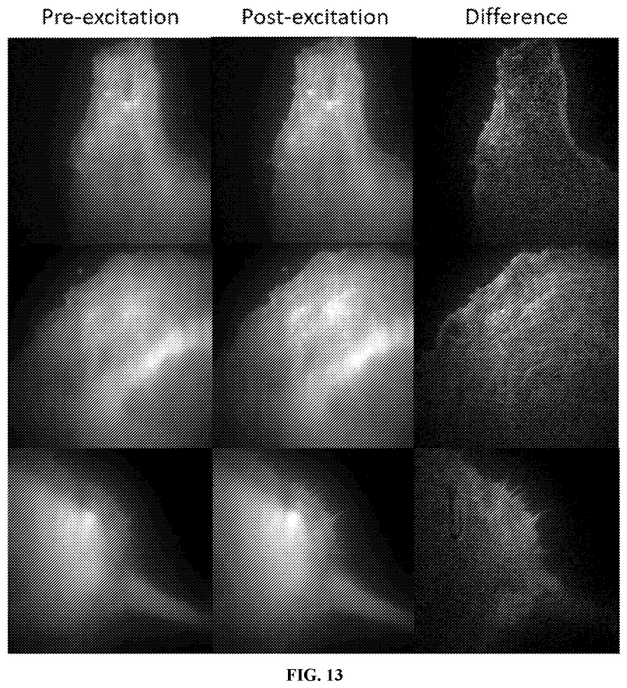

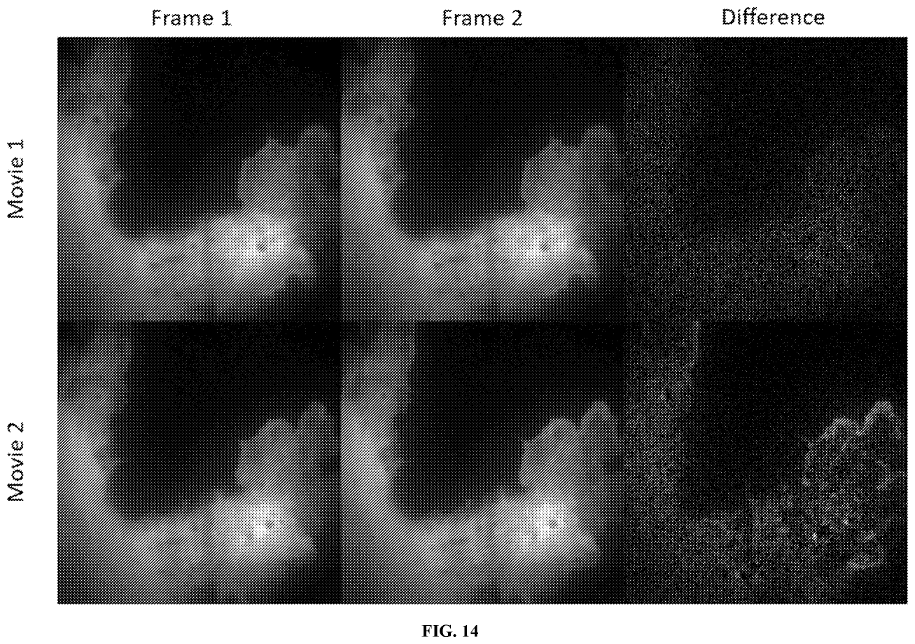

U2OS cells were maintained in DMEM (Thermo)+10% fetal bovine serum. Cells were split at 50-80% confluency every 2-3 days in 6-well plates after washing wish DPBS (Sigma) and trypsinizing for 2 minutes. For imaging, cells were seeded onto Delta T dishes (Fisher) for 24 hours. Then, cells were transfected using FuGene (Promega) according to the standard protocol. After 8 hours, media was changed to Fluoribrite DMEM (Thermo) and the dish was heated using a stage heater and objective heater set to 37° C. As shown in FIGS. 13-14, LILAC specifically labels U2OS actin structures post blue-light illumination.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A polypeptide comprising the amino acid sequence: AXXIXXXA(M)ₙGVADLIKKFE(X′)_{n′} (SEQ ID NO:1) or an amino acid sequence with at least 80% sequence identity to SEQ ID NO:1, wherein X is any amino acid, n and n′ are each independently selected from 0 and 1, and X′ is equal to SISKEE.

2. A polypeptide comprising the amino acid sequence: AXXI(M)ₙGVADLIKKFE(X′)_{n′} (SEQ ID NO: 14) or an amino acid sequence with at least 80% sequence identity to SEQ ID NO: 14, wherein X is any amino acid, n and n′ are each independently selected from 0 and 1, and X′ is equal to SISKEE.

3. The polypeptide of claim 1 or 2, wherein (M)ₙGVADLIKKFE(X′)ₙ (SEQ ID NO:47) or an amino acid sequence with at least 80% sequence identity to SEQ ID NO:47 is further defined as an actin-binding polypeptide, wherein X is any amino acid, n and n′ are each independently selected from 0 and 1, and X′ is equal to SISKEE.

4. The polypeptide of claim 1 or 3, wherein the polypeptide comprises the amino acid sequence AXXIXXXAMGVADLIKKFESISKEE (SEQ ID NO:2) or an amino acid sequence with at least 80% sequence identity to SEQ ID NO:2.

5. The polypeptide of claim 1 or 3, wherein the polypeptide comprises the amino acid sequence AEEIDEAAMGVADLIKKFESISKEE (SEQ ID NO:3) or an amino acid sequence with at least 80% sequence identity to SEQ ID NO:3.

6. The polypeptide of claim 1 or 3, wherein the polypeptide comprises the amino acid sequence AEEIDEAAMEVADLIKKFESISKEE (SEQ ID NO:4) or an amino acid sequence with at least 80% sequence identity to SEQ ID NO:4.

7. The polypeptide of claim 1 or 3, wherein the polypeptide comprises the amino acid sequence AEEIDEAAMGAADLIKKFESISKEE (SEQ ID NO:5) or an amino acid sequence with at least 80% sequence identity to SEQ ID NO:5.

8. The polypeptide of claim 1 or 3, wherein the polypeptide comprises the amino acid sequence AEEIDEAAMAVADLIKKFESISKEE (SEQ ID NO:6) or an amino acid sequence with at least 80% sequence identity to SEQ ID NO:6.

9. The polypeptide of claim 1 or 3, wherein the polypeptide comprises the amino acid sequence AENIDEAAMGVADLIKKFESISKEE (SEQ ID NO:7) or an amino acid sequence with at least 80% sequence identity to SEQ ID NO:7.

10. The polypeptide of claim 1 or 3, wherein the polypeptide comprises the amino acid sequence AENIDEAAMEVADLIKKFESISKEE (SEQ ID NO:8) or an amino acid sequence with at least 80% sequence identity to SEQ ID NO:8.

11. The polypeptide of claim 1 or 3, wherein the polypeptide comprises the amino acid sequence AENIDEAAMGAADLIKKFESISKEE (SEQ ID NO:9) or an amino acid sequence with at least 80% sequence identity to SEQ ID NO:9.

12. The polypeptide of claim 1 or 3, wherein the polypeptide comprises the amino acid sequence AENIDEAAMAVADLIKKFESISKEE (SEQ ID NO:10) or an amino acid sequence with at least 80% sequence identity to SEQ ID NO:10.

13. The polypeptide of claim 2-3, wherein the polypeptide comprises the amino acid sequence AEEIMGVADLIKKFESISKEE (SEQ ID NO:11) or an amino acid sequence with at least 80% sequence identity to SEQ ID NO:11.

14. The polypeptide of claim 2-3, wherein the polypeptide comprises the amino acid sequence AENIMGVADLIKKFESISKEE (SEQ ID NO: 12) or an amino acid sequence with at least 80% sequence identity to SEQ ID NO: 12.

15. The polypeptide of any one of claims 1-14, wherein the polypeptide further comprises GEFLATTLERIEKNFVITDPRLPDNPIIFASDSFLQLTEYSREEILGRNCRFLQGP ETDRATVRKIRDAIDNQTEVTVQLINYTKSGKKFWNLFHLQPMRDQKGDVQ YFIGVQLDGTEHVRDAAEREGVMLIKKT (SEQ ID NO:13) at the amino proximal position or an amino acid sequence with at least 70% sequence identity to SEQ ID NO: 13 at the amino proximal position.

16. The polypeptide of any one of claims 1-15, wherein the polypeptide comprises at least one substitution relative to the amino acid sequence.

17. The polypeptide of claim 16, wherein the polypeptide comprises a G128A substitution of SEQ ID NO: 13.

18. The polypeptide of claim 16 or 17, wherein the polypeptide comprises a 1132A substitution of SEQ ID NO:13.

19. The polypeptide of any one of claims 16-18, wherein the polypeptide comprises a N138E substitution of SEQ ID NO:24.

20. The polypeptide of any one of claims 16-19, wherein the polypeptide comprises a G145E or G145A substitution of SEQ ID NO:24.

21. The polypeptide of any one of claims 16-20, wherein the polypeptide comprises a V146A substitution of SEQ ID NO:24.

22. The polypeptide of any one of claims 16-21, wherein the polypeptide comprises a G141E or G141A substitution of SEQ ID NO:24.

23. The polypeptide of any one of claims 16-22, wherein the polypeptide comprises a V142A substitution of SEQ ID NO:24.

24. The polypeptide of any one of claims 1-23, wherein the polypeptide comprises the amino acid sequence of one of SEQ ID NOS: 15-46, or an amino acid sequence with at least 70% sequence identity to one of SEQ ID NOS: 15-46.

25. The polypeptide of claim 24, wherein the polypeptide comprises the amino acid sequence of one of SEQ ID NOS: 24, 26, 28, or 30, or an amino acid sequence with at least 70% sequence identity to one of SEQ ID NOS: 24, 26, 28, or 30.

26. The polypeptide of any one of claims 1-25, wherein the polypeptide is a photoswitch.

27. The polypeptide of any one of claims 1-26, wherein the polypeptide binds to actin upon photo-excitation.

28. The polypeptide of any one of claims 1-27, wherein the polypeptide has low affinity for actin in the dark.

29. The polypeptide of any one of claims 1-28, wherein the polypeptide has a length of 250 amino acids or less.

30. The polypeptide of any one of claims 1-29, wherein the polypeptide is conjugated to at least one heterologous molecule.

31. The polypeptide of claim 30, wherein the heterologous molecule comprises a heterologous polypeptide.

32. The polypeptide of claim 31, wherein the heterologous molecule is a fluorescent polypeptide such as GFP or a variant thereof.

33. The polypeptide of claim 30, wherein the heterologous molecule is a non-peptidic molecule.

34. The polypeptide of claim 33, wherein the heterologous molecule is a fluorescent labelling group such as fluorescein or rhodamine.

35. The polypeptide of any one of claims 30-34, wherein the heterologous molecule is coupled to the N- and/or C-terminus of the polypeptide.

36. The polypeptide of any one of claims 3-35, wherein the polypeptide further comprise one or more additional actin binding protein(s).

37. The polypeptide of claim 36, wherein at least one additional actin binding protein is at the N-terminus of the polypeptide.

38. The polypeptide of claim 36 or 37, wherein the additional actin binding protein(s) comprise LifeAct (SEQ ID NO:47), MGVADLIKKFESISKEE (SEQ ID NO:48), MEVADLIKKFESISKEE (SEQ ID NO:49), MGAADLIKKFESISKEE (SEQ ID NO: 50), MAVADLIKKFESISKEE (SEQ ID NO:51), vtrophin, or F-tractin.

39. A nucleic acid encoding the polypeptide of any one of claims 1-38.

40. An expression vector comprising the nucleic acid of claim 39.

41. A host cell comprising the polypeptide of any one of claims 1-38, the nucleic acid of claim 39, or the expression vector of claim 40.

42. The cell of claim 41, wherein the cell comprises a eukaryotic cell.

43. The cell of claim 42, wherein the cell comprises a fibroblast or muscle cell.

44. A method of making a cell comprising transferring the expression vector of claim 40 into a cell.

45. The method of claim 44, wherein the expression vector is transfected into the cell.

46. The method of claim 44 or 45, wherein the method further comprises isolating, purifying, imaging, or quantitating polypeptides expressed in the cell.

47. The method of claim 46, wherein the method further comprises isolating, purifying, imaging, or quantitating polypeptides expressed from the expression vector in the cell.

48. A method comprising contacting a cell with the polypeptide of any one of claims 1-35.

49. The method of claim 48, wherein the cell is fixed.

50. The method of claim 48 or 49, wherein the cell is a non-dividing cell.

51. A method of making a polypeptide comprising transferring the expression vector of claim 40 into a cell and incubating the cell under conditions that allow for the expression of the polypeptide.

52. The method of claim 51, wherein the expression vector is transfected into the cell.

53. The method of claim 51 or 52, wherein the method further comprises isolating, purifying, imaging, or quantitating polypeptides expressed in the cell.

54. The method of any one of claims 51-53, wherein the method further comprises isolating, purifying, imaging, or quantitating polypeptides expressed from the expression vector in the cell.

55. A solid support comprising the polypeptide of any one of claims 1-38 linked to or coated on the solid support.

56. The solid support of claim 55, wherein the solid support comprises a microscope slide.

57. The solid support of claim 56 or 57, wherein the polypeptide is linked to the solid support covalently or non-covalently.

58. The solid support of any one of claims 55-57, wherein the polypeptide is linked to the solid support through an antibody.

59. The solid support of claim 58, wherein the polypeptide comprises a heterologous molecule and the antibody comprises an antibody that specifically recognizes the heterologous molecule.

60. The solid support of claim 59, wherein the heterologous molecule comprises a fluorescent protein.

61. A method for labeling actin in a cell comprising transferring the expression vector of claim 40 into the cell or contacting the cell with the polypeptide of any one of claims 1-38 or the solid support of any one of claims 55-60.

62. The method of claim 61, wherein the cell comprises a eukaryotic cell.

63. The cell of claim 62, wherein the cell comprises a fibroblast or muscle cell.

64. The method of claim 62, wherein the cell comprises a cardiomyocyte.

65. The method of claim 61 or 62, wherein the method comprises transferring the expression vector into the cell and wherein the method further comprises quantitatively or qualitatively assaying for polypeptides expressed from the expression vector.

66. The method of claim 65, wherein the polypeptide comprises a fluorescent polypeptide.

67. The method of claim 66, wherein the method further comprises imaging and/or quantitating the fluorescent polypeptide.

68. The method of any one of claims 65-67, wherein the method further comprises performing an assay to detect or quantitate the polypeptide expressed from the expression vector.

69. The method of claim 68, wherein the assay comprises one or more of a western blot, an elisa, immunofluorescence, or an immunoassay.

70. The method of any one of claims 61-69, wherein labeling comprises photo-excitation conditional labeling of actin.

71. The method of any one of claims 61-70, wherein the cell is a fixed cell.

72. The method of any one of claims 61-70, wherein the cell is a non-fixed living cell.

73. The method of any one of claims 1-70 or 72, wherein the cell is a cell in cell culture.
